(19) United States
(12) Patent Application Publication
(10) Pub. No.: US 2004/0023870 A1
(43) Pub. Date: Feb. 5, 2004

(54) Methods of therapy and diagnosis using targeting of cells that express toll-like receptor proteins

(76) Inventors: Douglas Dedera, Castro Valley, CA (US); Peter C.R. Emtage, Sunnyvale, CA (US)

Correspondence Address: Renee S. Polizotto, 675 Almanor Avenue, Sunnyvale, CA, 94085, US

(21) Appl. No.: 10/327,491

(22) Filed: Dec. 19, 2002

Related U.S. Application Data
(63) Continuation-in-part of application No. 10/302,444, filed on Nov. 22, 2002.
Application No. 10/302,444 is a continuation-in-part of application No. 10/077,676, filed on Feb. 14, 2002.
Application No. 10/077,676 is a continuation-in-part of application No. 09/687,527, filed on Oct. 12, 2000, now abandoned.
Application No. 10/077,676 is a continuation-in-part of application No. 09/488,725, filed on Jan. 21, 2000.

(51) Int. Cl. A61K 39/395; A61K 38/17

(52) U.S. Cl. 514/12; 424/144.1

(57) ABSTRACT

Certain cells, including types of cancer cells such as B-cell lymphomas, T cell lymphomas, Hodgkin's disease and myeloid leukemias, are capable of expressing Toll-like Receptor 9 (TLR9) or Toll-like Receptor 10 (TLR10) mRNA. Immunotargeting using TLR9 or TLR10 polypeptides, nucleic acids encoding for TLR9 or TLR10 polypeptides and anti-TLR9 or anti-TLR10 antibodies provides a method of killing or inhibiting that growth of cancer cells that express the TLR9 or TLR1O protein. Methods of immunotherapy and diagnosis of disorders associated with TLR9 or TLR1O protein-expressing cells, such as B-cell lymphoma, T cell lymphoma, acute myeloid leukemia, Hodgkin's disease, B cell leukemia, chronic lymphocytic leukemia, chronic myelogenous leukemia and myelodysplastic syndromes, are described.

### CROSS REFERENCE TO RELATED APPLICATIONS

This application is a continuation-in-part application of U.S. application Ser. No. 10/302,444 filed on Nov. 22, 2002, entitled “Methods of Therapy and Diagnosis Using Targeting of Cells that Express Toll-like Receptor Proteins,” Attorney Docket No. HYS-49CP, which in turn is a continuation-in-part application of U.S. application Ser. No. 10/077,676 filed on Feb. 14, 2002, entitled “Methods of Therapy and Diagnosis Using Targeting of Cells that Expressing Toll-Like Receptor 9 Protein”, Attorney Docket No. HYS-49, which in turn is a continuation-in-part application of U.S. application Ser. No. 09/687,527 filed on Oct. 12, 2000, entitled “Full Length Novel Nucleic Acids and Polypeptides”, Attorney Docket No. 795, and U.S. application Ser. No. 09/488,725 filed on Jan. 21, 2000, entitled “Novel Contigs Obtained from Various Libraries,” Attorney Docket No. 784. This and all other U.S. Patents and Patent Applications cited herein are hereby incorporated by reference in their entirety.

### TECHNICAL FIELD

This invention relates to compositions and methods for targeting Toll-like Receptor 9 (TLR9) protein- and Toll-like Receptor 10 (TLR10) protein-expressing cells and their use in the therapy and diagnosis of various pathological states, including cancer, autoimmune disease, organ transplant rejection, and allergic reactions.

### BACKGROUND

Antibody therapy for cancer involves the use of antibodies, or antibody fragments, against a tumor antigen to target antigen-expressing cells. Antibodies, or antibody fragments, may have direct or indirect cytotoxic effects or may be conjugated or fused to cytotoxic moieties. Direct effects include the induction of apoptosis, the blocking of growth factor receptors, and anti-idiotype antibody formation. Indirect effects include antibody-dependent cell-mediated cytotoxicity (ADCC) and complement-mediated cellular cytotoxicity (CMCC). When conjugated or fused to cytotoxic moieties, the antibodies, or fragments thereof, provide a method of targeting the cytotoxicity towards the tumor antigen expressing cells. (Green, et al., Cancer Treatment Reviews, 26:269-286 (2000), this and all other references cited herein are hereby incorporated by reference in their entirety.).

Because antibody therapy targets cells expressing a particular antigen, there is a possibility of cross-reactivity with normal cells or tissue. Although some cells, such as hematopoietic cells, are readily replaced by precursors, cross-reactivity with many tissues can lead to detrimental results. Thus, considerable research has gone towards finding tumor-specific antigens. Such antigens are found almost exclusively on tumors or are expressed at a greater level in tumor cells than the corresponding normal tissue. Tumor-specific antigens provide targets for antibody targeting of cancer, or other disease-related cells, expressing the antigen. Antibodies specific to such tumor-specific antigens can be conjugated to cytotoxic compounds or can be used alone in immunotherapy. Immunotoxins target cytotoxic compounds to induce cell death. For example, anti-CD22 antibodies conjugated to deglycosylated ricin A may be used for treatment of B cell lymphoma that has relapsed after conventional therapy (Amlot, et al., Blood 82:2624-2633 (1993)) and has demonstrated encouraging responses in initial clinical studies.

Immunotherapy provides a method of harnessing the immune system to treat various pathological states, including cancer, autoimmune disease, transplant rejection, hyperproliferative conditions, and allergic reactions.

The immune system functions to eliminate organisms or cells that are recognized as non-self, including microorganisms, neoplasms and transplants. A cell-mediated host response to tumors includes the concept of immunologic surveillance, by which cellular mechanisms associated with cell-mediated immunity, destroy newly transformed tumor cells after recognizing tumor-associated antigens (antigens associated with tumor cells that are not apparent on normal cells). Furthermore, a humoral response to tumor-associated antigens enables destruction of tumor cells through immunological processes triggered by the binding of an antibody to the surface of a cell, such as antibody-dependent cellular cytotoxicity (ADCC) and complement mediated lysis.

Recognition of an antigen by the immune system triggers a cascade of events including cytokine production, B-cell proliferation, and subsequent antibody production. Often tumor cells have reduced capability of presenting antigen to effector cells, thus impeding the immune response against a tumor-specific antigen. In some instances, the tumor-specific antigen may not be recognized as non-self by the immune system, preventing an immune response against the tumor-specific antigen from occurring. In such instances, stimulation or manipulation of the immune system provides effective techniques of treating cancers expressing one or more tumor-specific antigens.

For example, Rituximab (Rituxan®) is a chimeric antibody directed against CD20, a B cell-specific surface molecule found on >95% of B-cell non-Hodgkin's lymphoma (Press, et al., Blood 69:584-591 (1987); Malony, et al., Blood 90:2188-2195 (1997)). Rituximab induces ADCC and inhibits cell proliferation through apoptosis in malignant B cells in vitro (Maloney, et al., Blood 88:637a (1996)). Rituximab is currently used as a therapy for advanced stage or relapsed low-grade non-Hodgkin's lymphoma, which has not responded to conventional therapy.

Active immunotherapy, whereby the host is induced to initiate an immune response against its own tumor cells can be achieved using therapeutic vaccines. One type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89:3129-3135 (1997)). Another type of vaccine uses antigen-presenting cells (APCs), which present antigen to naive T cells during the recognition and effector phases of the immune response. Dendritic cells, one type of APC, can be used in a cellular vaccine in which the dendritic cells are isolated from the patient, co-cultured with tumor antigen and then reinfused as a cellular vaccine (Hsu, et al., Nat. Med. 2:52-58 (1996)). Immune responses can also be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002)).

Toll and toll-like receptors are type I transmembrane proteins with extracellular leucine-rich repeat motifs and an intracellular signaling domains. The toll-like receptors make up a family of human receptors which have common structural features with the Drosophila Toll (dToll) receptor molecule. They are found on the surface of several types of hematopoietic cells. Human Toll-like receptors are also expressed on antigen presenting cells, such as monocytes and dendritic cells (WO 01/55386 A1). Two human colon cancer cell lines (DLD and LoVo) showed expression of the toll-like receptor subtype TLR-2, whereas the toll-like receptor subtype TLR-4 was expressed in human hepatocellular carcinoma (PLC/PRF/5) and acute myeloid leukemia (KG-1) cells (Yoshioka, et al., J. Int. Med. Res. 29:409-420 (2001)).

Both dToll and human toll-like receptors are thought to act as pattern recognition receptors for bacteria and other microorganisms, and play a role in immune surveillance mechanisms and innate immunity. Toll-like receptors can trigger pro-inflammatory cytokine production and induce expression of cell surface co-stimulatory receptors for T-cell activation. Some human toll-like receptors may be involved in co-ordination of interactions between immune cells resulting in an integrated immune response to infection. TLR2 and TLR4 have been shown to mediate host responses to gram positive and gram negative bacteria through recognition of specific bacterial wall components. TLR4 mediates responses to certain viral proteins such as respiratory syncytial virus. Toll-like receptors may also form heterodimeric functional complexes and share in common signal transduction pathways with IL-1 receptors. Activation of TLR2 and TLR4 leads to the activation of NFKB via an adapter protein MyD88 and recruitment of the IL-1 receptor-associated kinases (IRAKs) (WO 01/55386 A1; Henneke and Golenbock, Nature Immunology 2:828-830 (2001)) Toll-like Receptor 9 (TLR9) was shown to mediate the cellular response to bacterial deoxy-cytidylate-phosphate-deoxyguanylate (CpG) DNA, suggesting that vertebrate systems have evolved a specific Toll-like receptor that distinguishes bacterial DNA from self-DNA (Hemml, et al., Nature 408:740-744 (2000); Bauer, et al., Proc. Natl. Acad. Sci. 98:9237-9242 (2001); Takeshita, et al., J. Immunol. 167:3555-3558 (2001); Wagner, Immunity 14:499-502 (2001)). In addition, CpG DNA may be recognized by autoantibodies inducing an autoimmune response (Ichikawa et al., J. Immunol. 169:2781-2787 (2002); Leadbetter et al., Nature 416;603-607 (2002), both are herein incorporated by reference). Thus, agents that block TLR9 and other Toll-like receptor proteins may be useful in treating autoimmune disorders. A new member of the TLR family was recently discovered, TLR10, which is most closely related to TLR1 and TLR6 (Chuang and Ulevitch, Biochim, Biophys, Acta 1518:157-161 (2001), herein incorporated by reference). The cytoplasmic domains of TLR1 and TLR6 have been demonstrated to form functional pairs with TLR2, thus TLR10 may also interact with TLR2 (Homung et al., J. Immunol. 168:4531-4535 (2002); Ozinsky et al., Proc. Natl. Acad. Sci. USA 97:13766 (2000), both of which are herein incorporated by reference). These findings suggest that toll and toll-like receptors may play a role in immune defense mechanisms to counteract microbial infection.

Thus, there exists a need in the art to identify and develop agents, such as peptide fragments, nucleic acids, or antibodies that provide therapeutic compositions and diagnostic methods for treating and identifying cancer, hyperproliferative disorders, autoimmune diseases, and organ transplant rejection.

### SUMMARY OF THE INVENTION

The invention provides therapeutic and diagnostic methods of targeting cells expressing the Toll-like Receptor 9 (TLR9) or Toll-like Receptor 10 (TLR10) protein by using targeting elements such as TLR9 or TLR10 polypeptides, nucleic acids encoding TLR9 or TLR10 protein, and anti-TLR9 or anti-TLR10 antibodies, including fragments or other modifications thereof. The TLR9 and TLR10 proteins are highly expressed in certain hematopoeitic-based cancer cells relative to their expression in healthy cells. Thus, targeting of cells that express TLR9 or TLR10 will have a minimal effect on healthy tissues while destroying or inhibiting the growth of the hematopoeitic-based cancer cells. Similarly, non-hematopoeitic type tumors (solid tumors) can be targeted if they bear the TLR9 or TLR10 antigen. For example, inhibition of growth and/or destruction of TLR9 or TLR10-expressing cancer cells results from targeting such cells with anti-TLR9 or anti-TLR10 antibodies, respectively. One embodiment of the invention is a method of destroying TLR9 or TLR10-expressing cells by conjugating anti-TLR9 or anti-TLR10 antibodies with cytocidal materials such as radioisotopes or other cytotoxic compounds, respectively.

The present invention provides a variety of targeting elements and compositions. One such embodiment is a composition comprising an anti-TLR9 or anti-TLR10 antibody preparation. Exemplary antibodies include a single anti-TLR9 or anti-TLR10 antibody, a combination of two or more anti-TLR9 or anti-TLR10 antibodies, a combination of an anti-TLR9 or anti-TLR10 antibody with a non-TLR9 or non-TLR10 antibody, a combination of an anti-TLR9 or anti-TLR10 antibody and a therapeutic agent, a combination of an anti-TLR9 or anti-TLR10 antibody and a cytocidal agent, a bispecific anti-TLR9 or anti-TLR10 antibody, Fab TLR9 or TLR10 antibodies or fragments thereof, including any fragment of an antibody that retains one or more complementary determining regions (CDRs) that recognize TLR9 or TLR10, humanized anti-TLR9 or anti-TLR10 antibodies that retain all or a portion of a CDR that recognizes TLR9 or TLR10, anti-TLR9 or anti-TLR10 conjugates, and anti-TLR9 or anti-TLR10 antibody fusion proteins.

Another targeting embodiment of the invention is a vaccine comprising a TLR9 or TLR10 polypeptide, or a fragment or variant thereof and optionally comprising a suitable adjuvant.

Yet another targeting embodiment is a composition comprising a nucleic acid encoding TLR9 or TLR10, or a fragment or variant thereof, optionally within a recombinant vector. A further targeting embodiment of the present invention is a composition comprising an antigen-presenting cell transformed with a nucleic acid encoding TLR9 or TLR10, or a fragment or variant thereof, optionally within a recombinant vector. The present invention further provides a method of targeting TLR9- or TLR10-expressing cells, which comprises admninistering a targeting element or composition in an amount effective to target TLR9-expressing cells. Any one of the targeting elements or compositions described herein may be used in such methods, including an anti-TLR9 or anti-TLR10 antibody preparation, a vaccine comprising a TLR9 or TLR10 polypeptide, or a fragment or variant thereof or a composition of a nucleic acid encoding TLR9 or TLR10, or a fragment or variant thereof, optionally within a recombinant vector or a composition of an antigen-presenting cell transformed with a nucleic acid encoding TLR9 or TLR10, or fragment or variant thereof, optionally within a recombinant vector.

The invention also provides a method of inhibiting the growth of hematopoetic-based, TLR9- or TLR10-expressing cancer cells, which comprises administering a targeting element or a targeting composition in an amount effective to inhibit the growth of said hematopoetic-based cancer cells. Any one of the targeting elements or compositions described herein may be used in such methods, including an anti-TLR9 or anti-TLR10 antibody preparation, a vaccine comprising a TLR9 or TLR10 polypeptide, fragment, or variant thereof, composition of a nucleic acid encoding TLR9 or TLR10, or fragment or variant thereof, optionally within a recombinant vector, or a composition of an antigen-presenting cell transformed with a nucleic acid encoding TLR9 or TLR10, or fragment or variant thereof, optionally within a recombinant vector.

The present invention further provides a method of treating disorders associated with the proliferation of TLR9- or TLR10-expressing cells in a subject in need thereof, comprising the step of administering a targeting element or targeting composition in a therapeutically effective amount to treat disorders associated with TLR9- or TLR10-expressing cells. Any one of the targeting elements or compositions described herein may be used in such methods, including an anti-TLR9 or anti-TLR10 antibody preparation, a vaccine comprising a TLR9 or TLR10 polypeptide, fragment, or variant thereof, a composition of a nucleic acid encoding TLR9 or TLR10, or fragment or variant thereof, optionally within a recombinant vector, or a composition of an antigen-presenting cell comprising a nucleic acid encoding TLR9, or fragment or variant thereof, optionally within a recombinant vector. Examples of disorders associated with the proliferation of TLR9- or TLR10-expressing cells include cancers, such as non-Hodgkin's B-cell lymphomas, B-cell leukemias, chronic lymphocytic leukemia, multiple myeloma, acute and chronic myeloid leukemia; myelodysplastic syndromes; T cell lymphomas, acute myelogenous leukemia, acute myelomonocytic leukemia, acute lymphoblastic leukemia, chronic myelogenous leukemia, lymphosarcoma leukemia, malignant lymphoma, B cell large cell lymphoma, X-linked lymphoproliferative disorders; Epstein Barr Virus-related conditions such as mononucleosis; and autoimmune disorders. Non-hematopoietic tumors that bear the TLR9 or TLR10 antigen, such as prostate, breast, colon, and squamous cell carcinoma, as well as other cancers of epithelial and squamous cell origin, can also be targeted. The invention further provides a method of modulating the immune system by either suppression or stimulation of growth factors and cytokines, by administering the targeting elements or compositions of the invention. The invention also provides a method of modulating the immune system through activation of immune cells (such as natural killer cells, T cells, B cells and myeloid cells), through the suppression of activation, or by stimulating or suppressing proliferation of these cells by TLR9 or TLR10 peptide fragments or TLR9 or TLR10 antibodies.

The present invention thereby provides a method of treating immune-related disorders by suppressing the immune system in a subject in need thereof, by administering the targeting elements or compositions of the invention. Such immune-related disorders include but are not limited to autoimmune disease and organ transplant rejection.

The present invention also provides a method of diagnosing disorders associated with TLR9- or TLR10-expressing cells comprising the step of measuring the expression patterns of TLR9 or TLR10 protein and/or mRNA, respectively. Yet another embodiment of a method of diagnosing disorders associated with TLR9- or TLR10-expressing cells comprising the step of detecting TLR9 or TLR10 expression using anti-TLR9 or anti-TLR10 antibodies, respectively. Such methods of diagnosis include compositions, kits and other approaches for determining whether a patient is a candidate for TLR9 or TLR10 immunotherapy.

The present invention also provides a method of enhancing the effects of therapeutic agents and adjunctive agents used to treat and manage disorders associated with TLR9- or TLR10-expressing cells, by administering TLR9 or TLR10 preparations with therapeutic and adjuvant agents commonly used to treat such disorders.

### BRIEF DESCRIPTION OF THE DRAWING

FIGS. 1 (A-D) depicts the nucleic acid sequence of a cDNA encoding a TLR9 polypeptide and the amino acid sequence of the encoded polypeptide.

FIGS. 2 (A-C) depicts the nucleic acid sequence of a cDNA encoding a TLR10 polypeptide and the amino acid sequence of the encoded polypeptide.

### DETAILED DESCRIPTION OF THE INVENTION

The present invention relates to methods of targeting cells that express TLR9 or TLR10 using targeting elements, such as TLR9 or TLR10 polypeptides, nucleic acids encoding TLR9 or TLR10, anti-TLR9 or anti-TLR10 antibodies, including fragments or other modifications of any of these elements.

The present invention provides a novel approach for diagnosing and treating diseases and disorders associated with TLR9- or TLR10-expressing cells. The method comprises administering an effective dose of targeting preparations such as vaccines, antigen presenting cells, or pharmaceutical compositions comprising the targeting elements, TLR9 or TLR10 polypeptides, nucleic acids encoding TLR9 or TLR10, anti-TLR9 or anti-TLR10 antibodies, described below. Targeting of TLR9 or TLR10 on the cell membranes of TLR9- or TLR10-expressing cells, respectively, is expected to inhibit the growth of or destroy such cells. An effective dose will be the amount of such targeting TLR9 or TLR10 preparations necessary to target the TLR9 or TLR10 on the cell membrane and inhibit the growth of or destroy the TLR9- or TLR10-expressing cells and/or metastasis.

A further embodiment of the present invention is to enhance the effects of therapeutic agents and adjunctive agents used to treat and manage disorders associated with TLR9- or TLR10-expressing cells, by administering TLR9 or TLR10 preparations, respectively, with therapeutic and adjuvant agents commonly used to treat such disorders. Chemotherapeutic agents useful in treating neoplastic disease and antiproliferative agents and drugs used for immunosuppression include alkylating agents, such as nitrogen mustards, alkyl sulfonates, nitrosoureas, triazenes; antimetabolites, such as folic acid analogs, pyrimidine analogs, and purine analogs; natural products, such as vinca alkaloids, epipodophyllotoxins, antibiotics, and enzymes; miscellaneous agents such as polatinum coordination complexes, substituted urea, methyl hydrazine derivatives, and adrenocortical suppressant; and hormones and antagonists, such as adrenocorticosteroids, progestins, estrogens, androgens, and anti-estrogens (Calebresi and Parks, pp. 1240-1306 in, Eds. A. G Goodman, L. S. Goodman, T. W. Rail, and F. Murad, The Pharmacological Basis of Therapeutics, Seventh Edition, MacMillan Publishing Company, New York, (1985)).

Adjunctive therapy used in the management of such disorders includes, for example, radiosensitizing agents, coupling of antigen with heterologous proteins, such as globulin or beta-galactosidase, or inclusion of an adjuvant during immunization.

High doses may be required for some therapeutic agents to achieve levels to effectuate the target response, but may often be associated with a greater frequency of dose-related adverse effects. Thus, combined use of the immunotherapeutic methods of the present invention with agents commonly used to treat TLR9 or TLR10 protein-related disorders allows the use of relatively lower doses of such agents resulting in a lower frequency of adverse side effects associated with long-term administration of the conventional therapeutic agents. Thus another indication for the immunotherapeutic methods of this invention is to reduce adverse side effects associated with conventional therapy of disorders associated with TLR9-or TLR10-expressing cells.

Definitions

The term “fragment” of a nucleic acid refers to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1 or 3. A polypeptide “fragment” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity. The term “immunogenic” refers to the capability of the natural, recombinant or synthetic TLR9- or TLR10 peptide, or any peptide thereof, to induce a specific immune response in appropriate animals or cells and to bind with specific antibodies.

The term “variant” (or “analog”) refers to any polypeptide differing from naturally occurring polypeptides by amino acid insertions, deletions, and substitutions, created using, e g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Immunotargeting of TLR9 or TLR10

TLR9 polypeptides and polynucleotides encoding such polypeptides are disclosed in co-owned U.S. patent application Ser. No. 09/687,527. These and all other U.S. patents cited herein are hereby incorporated by reference in their entirety. U.S. Patent application Ser. No. 09/687,527 relates, in general, to novel isolated polypeptides, novel isolated polynucleotides encoding such polypeptides, including recombinant DNA molecules, cloned genes or degenerate variant thereof, especially naturally occurring variants such as allelic variants, antisense polynucleotide molecules, and antibodies that specifically recognize one or more epitopes present on such polypeptides, as well as hybridomas producing such antibodies. TLR10 polypeptides and polynucleotides encoding such polypeptides are disclosed in co-owned U.S. patent application Ser. No. 09/488,725, incorporated herein by reference, which relates, in general, to a collection or library of at least one novel nucleic acid sequences, specifically contigs, assembled from expressed sequence tags (ESTs). WO 01/55386 discloses a novel toll-like receptor and its use in screening for novel pharmacotherapeutic agents with immunomodulatory activity. More specifically, WO 01/55386 discloses isolated toll-like receptor polypeptides, polynucleotides encoding for the toll-like receptor polypeptide, expression vectors comprising such polynucleotides, hosts cells comprising such expression vectors, antibodies specific for the toll-like receptor polypeptide, methods for identification of compounds that modulate toll-like receptor activity, and methods of treating disorders responsive to toll-like receptor modulation. WO 99/20756 discloses human Toll homolog polypeptides, polynucleotides encoding for the human toll homologs, expression vectors comprising such polynucleotides, host cells comprising such expression vectors, antibodies specific for the human toll homolog polypeptides, antibodies that specifically bind to a human TLR2 receptor, and methods for treating septic shock using anti-toll homolog antibodies.

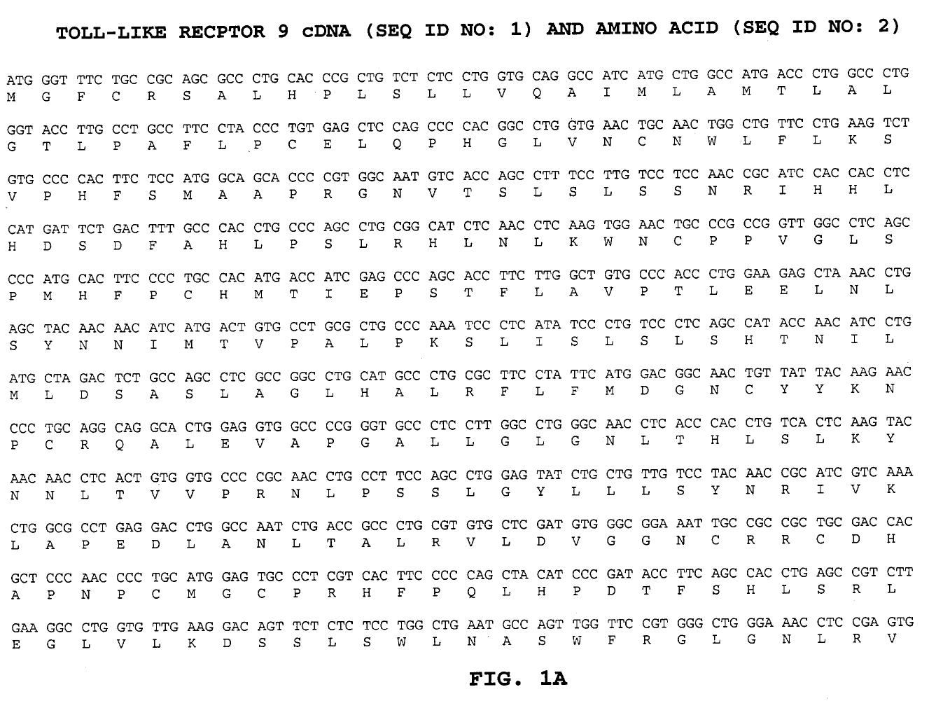

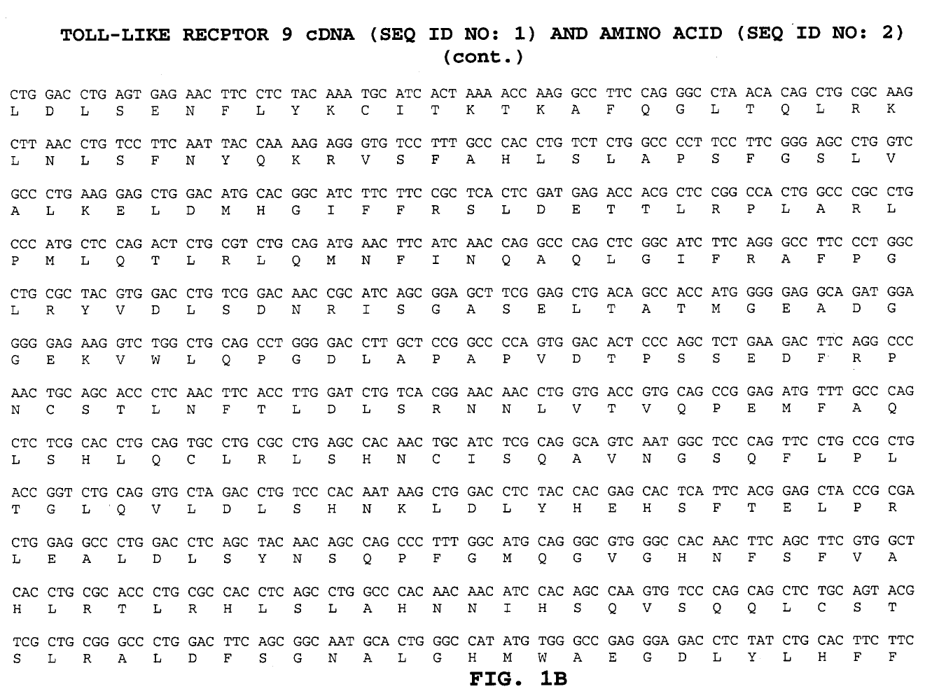

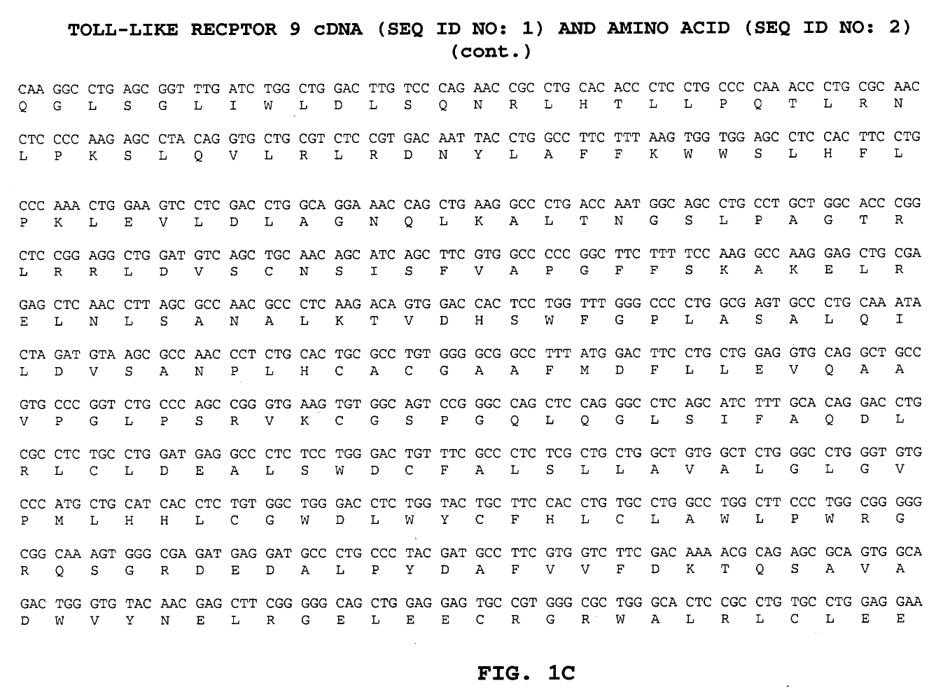

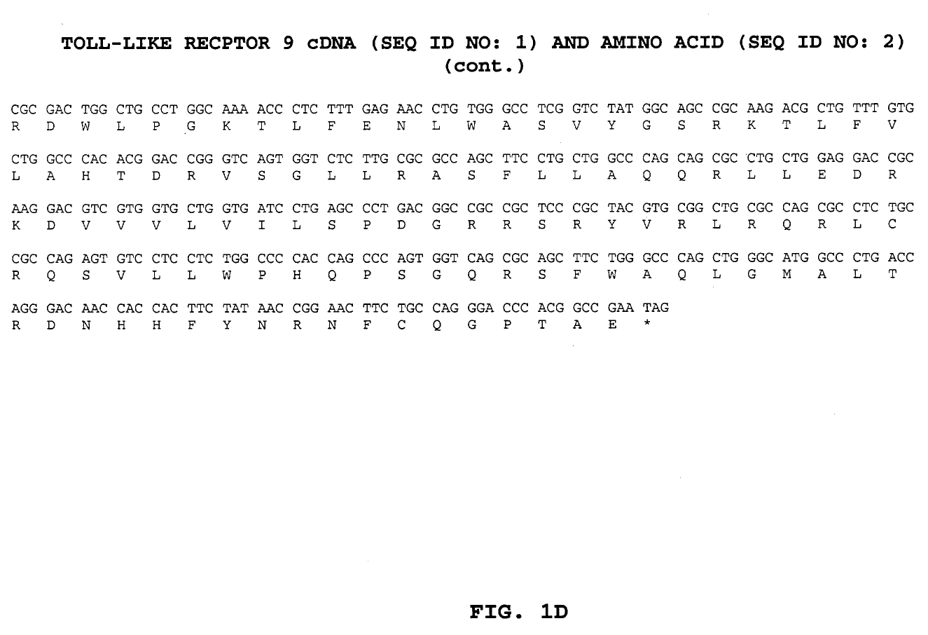

The amino acid sequence of an exemplary TLR9 polypeptide and the nucleic acid sequence of the cDNA encoding the polypeptide are provided in FIGS. 1A-D (SEQ ID NO: 1 and 2, respectively). Members of the Toll-like family of receptors were shown to be are expressed on antigen presenting cells, such as monocytes and dendritic cells (WO 01/55386 A1), human colon cancer cell lines (DLD and LoVo), human hepatocellular carcinoma (PLC/PRF/5) and acute myeloid leukemia (KG-1) cells (Yoshioka, et al., J. Int. Med. Res. 29:409-420 (2001)). Table 1 shows that TLR9 was found to be expressed at high levels in the B cell lymphoma cell lines CA-46, RL, GA-10 and HT, moderate levels in promyelomonocytic cell line HL-60 and the acute myeloid leukemia cell line AML-193, and at low levels in acute myeloid leukemia KG-1 cell line. These results demonstrate that TLR9 mRNA is highly expressed in cell lines derived from B cell lymphomas and myeloid leukemias. The results in Table 2 show that levels of mRNA expression were low to moderate in B cell lymphoma tissue (5348, 6879, 6796, 5856, 22601), Hodgkin's disease, acute myeloid leukemia and healthy peripheral blood monocytes (CD-14+). Medium levels of expression were observed in non-cancerous tonsilar lymph nodes, healthy peripheral blood B cells (CD19+ cells), and lung tissue. These findings demonstrate TLR9 mRNA expression in different Non-Hodgkin's B cell lymphoma tissues and cell lines, and indicate that TLR9 may be used as an immunotherapeutic antibody target and a diagnostic marker for certain cell types or disorders (e.g., B-cell lymphomas, T cell lymphomas, myeloid leukemia, Hodgkin's disease). The results in Table 3 show that TLR9 is expressed in the following leukemias and lymphomas: acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, and lymphosarcoma cell leukemia. Thus, targeting cells expressing TLR9 will be useful in treating these and other hematopoietic cell-based diseases. TLR9 is also expressed in solid tumors, such as prostate, breast, colon and squamous cell carcinoma (see Table 4). Since TLR9 is expressed in these epithelial and squamous cell cancers, it is likely that TLR9 will be expressed in other cancers of epithelial and squamous cell origin. Immunohistochemical (IHC) analysis showed that TLR9 is also expressed in autoimmune disorders, including systemic lupus erythematosus; Hasimoto thyroiditis, Sjörgen's syndrome, and pericarditis lupus (see Table 5). Therefore, targeting of TLR9 may be useful in treating these and other autoimmune disorders. Finally, IHC analysis showed that TLR9 is expressed in rejected transplanted heart, kidney and liver tissues, whereas normal tissues do not express TLR9 (see Table 6). Thus, targeting TLR9 will be useful in reducing and/or eliminating tissue rejection after transplantation.

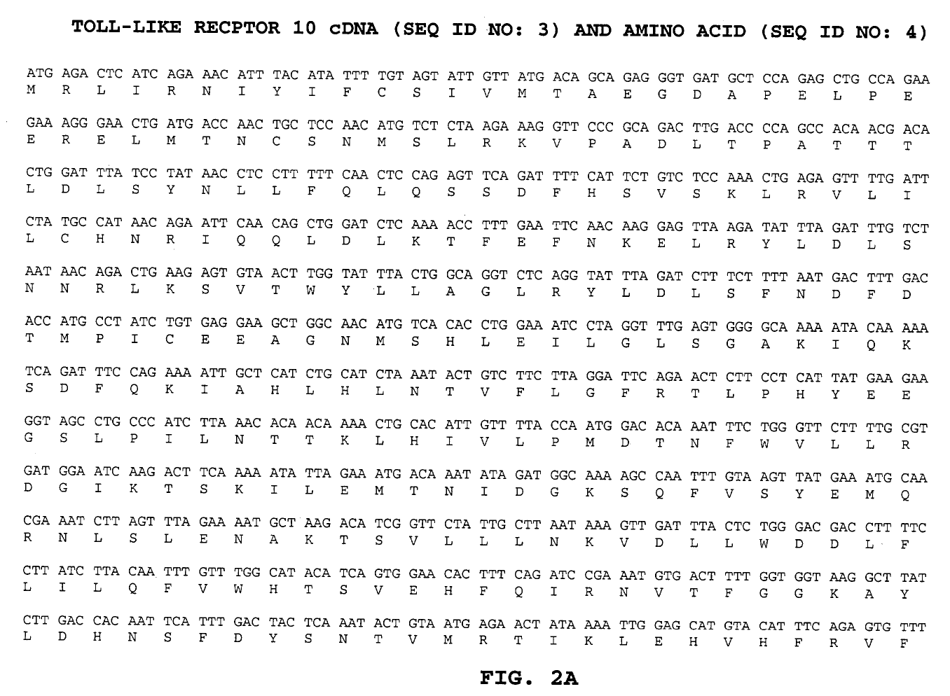

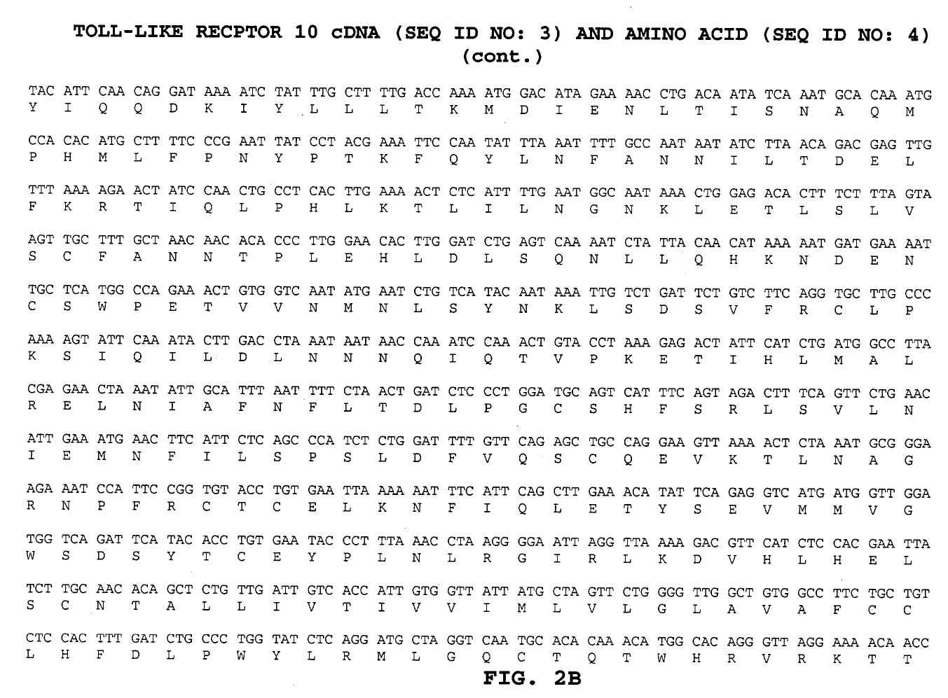

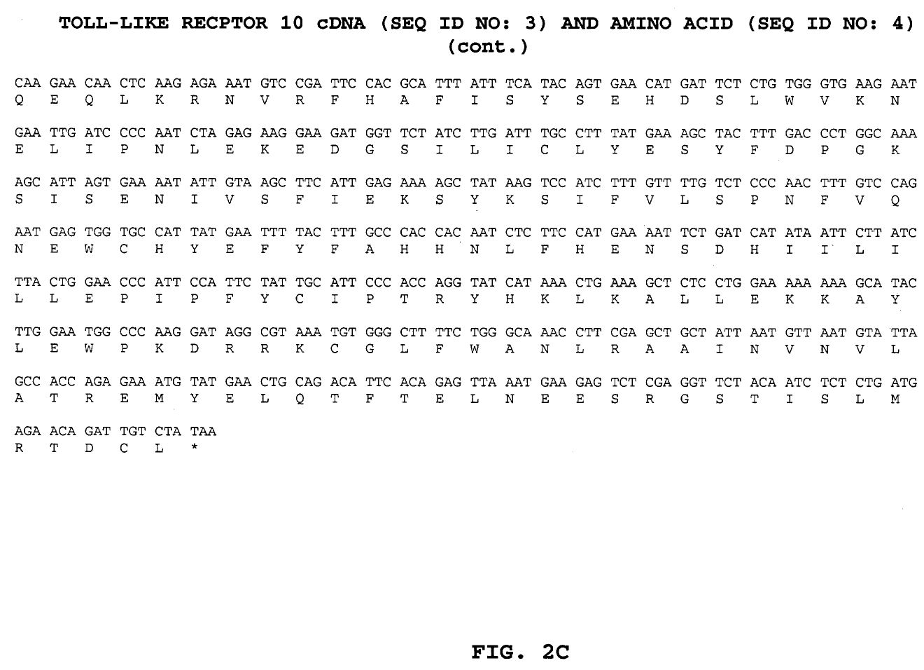

The amino acid sequence of an exemplary TLR10 polypeptide and the nucleic acid sequence of the cDNA encoding the polypeptide are provided in FIGS. 2A-C (SEQ ID NO: 3 and 4, respectively). Table 7 shows that TLR10 was found to be expressed at high levels in the B cell lymphoma cell lines CA-46 and RA1, moderately expressed in the B cell lymphoma cell lines DB, HT, and RL. These results demonstrate that TLR10 mRNA is hightly expressed in cell lines derived from B cell lymphomas. The results in Table 8 show that levels of mRNA expression were high in follicular lymphoma tissue (L5856 and L5348), moderate in diffuse large B cell lymphoma tissue (L6879 and L22601), and anaplastic large T cell lymphoma tissue (L5664), and low in acute myeloid leukemia tissue (AML565). These findings demonstrate TLR10 mRNA expression in different Non-Hodgkin's B cell lymphoma tissues and cell lines, and indicate that TLR10 may be used as an immunotherapeutic antibody target and a diagnostic marker for certain cell types or disorders (e.g., B-cell lymphomas, T cell lymphomas, and myeloid leukemia). The results in Table 9 show that TLR10 is expressed in the following leukemias and lymphomas: acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, and lymphosarcoma cell leukemia. Thus, targeting cells expressing TLR10 will be useful in treating these and other hematopoietic diseases. TLR10 is also expressed in solid tumors, such as prostate, breast, colon and squamous cell carcinoma (see Table 10). Since TLR10 is expressed in these epithelial and squamous cell cancers, it is likely that TLR10 will be expressed in other cancers of epithelial and squamous cell origin. IHC analysis showed that TLR10 is also expressed in autoimmune disorders, including systemic lupus erythematosus; Hasimoto thyroiditis, Sjörgen's syndrome, and pericarditis lupus (see Table 11). Therefore, targeting TLR10 may be useful in treating these and other autoimmune disorders. Finally, IHC analysis showed that TLR10 is expressed in rejected transplanted heart, kidney and liver tissues, whereas normal tissues do not express TLR10 (see Table 12). Thus, targeting TLR10 will be useful in reducing and/or eliminating tissue rejection after transplantation.

A. Targeting Using TLR9 or TLR10 Vaccines

The use of toll-like receptor proteins as adjuvants in vaccine preparations has been previously described (WO01/55386; Kovarik and Siegrist, Arch. Immunol. Ther. Exp. (Warsz) 49:209-215 (2001); Azuma and Seya, Int. Immunopharmacol. 1:1249-1259 (2001)). Thus one embodiment the present invention provides a vaccine comprising a TLR9 or TLR10 polypeptide to stimulate the immune system against TLR9 or TLR10, thus targeting TLR9- or TLR10-expressing cells, respectively. Use of a tumor antigen in a vaccine for generating cellular and humoral immunity for the purpose of anti-cancer therapy is well known in the art. For example, one type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89: 3129-3135 (1997)). U.S. Pat. No. 6,312,718 describes methods for inducing immune responses against malignant B cells, in particular lymphoma, chronic lymphocytic leukemia, and multiple myeloma. The methods described therein utilize vaccines that include liposomes having (1) at least one B-cell malignancy-associated antigen, (2) IL-2 alone, or in combination with at least one other cytokine or chemokine, and (3) at least one lipid molecule. Methods of vaccinating against TLR9 or TLR10 typically employ a TLR9 or TLR10 polypeptide, including fragments, analogs and variants.

As another example, dendritic cells, one type of antigen-presenting cell, can be used in a cellular vaccine in which the dendritic cells are isolated from the patient, co-cultured with tumor antigen and then reinfused as a cellular vaccine (Hsu, et al., Nat. Med. 2:52-58 (1996)).

B. Targeting Using TLR9 or TLR10 Nucleic Acids

1. Direct Delivery of Nucleic Acids

However, in some embodiments, a nucleic acid encoding TLR9 or TLR10, or encoding a fragment, analog or variant thereof, within a recombinant vector is utilized. Such methods are known in the art. For example, immune responses can be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002)). TLR9 or TLR10 viral vectors are particularly useful for delivering TLR9- or TLR10-encoding nucleic acids to cells, respectively. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. Of course, non-viral vectors, such as liposomes or even naked DNA, are also useful for delivering TLR9- or TLR10-encoding nucleic acids to cells.

Combining this type of therapy with other types of therapeutic agents or treatments such as chemotherapy or radiation is also contemplated.

2. TLR9 or TLR10 Nucleic Acids Expressed in Cells

In some embodiments, a vector comprising a nucleic acid encoding the TLR9 or TLR10 polypeptide (including a fragment, analog or variant) is introduced into a cell, such as a dendritic cell or a macrophage. When expressed in an antigen-presenting cell, TLR9 or TLR10 antigens are presented to T cells eliciting an immune response against TLR9 or TLR10. Such methods are also known in the art. Methods of introducing tumor antigens into antigen presenting cells and vectors useful therefor are described in U.S. Pat. No. 6,300,090. The vector encoding TLR9 or TLR10 may be introduced into the antigen presenting cells in vivo. Alternatively, antigen-presenting cells are loaded with TLR9 or TLR10 or a nucleic acid encoding TLR9 or TLR10 ex vivo and then introduced into a patient to elicit an immune response against TLR9 or TLR10, respectively. In another alternative, the cells presenting TLR9 or TLR10 antigen are used to stimulate the expansion of anti-TLR9 or anti-TLR10 cytotoxic T lymphocytes (CTL) ex vivo followed by introduction of the stimulated CTL into a patient. (U.S. Pat. No. 6,306,388)

Combining this type of therapy with other types of therapeutic agents or treatments such as chemotherapy or radiation is also contemplated.

C. Anti-TLR9 and Anti-TLR10 Antibodies

Alternatively, immunotargeting involves the administration of components of the immune system, such as antibodies, antibody fragments, or primed cells of the immune system against the target. Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens.

TLR9 or TLR10 antibodies (including humanized or human monoclonal antibodies or fragments or other modifications thereof, optionally conjugated to cytotoxic agents) may be introduced into a patient such that the antibody binds to TLR9 or TLR10 expressed by cancer cells and mediates the destruction of the cells and the tumor and/or inhibits the growth of the cells or the tumor. Without intending to limit the disclosure, mechanisms by which such antibodies can exert a therapeutic effect may include complement-mediated cytolysis, antibody-dependent cellular cytotoxicity (ADCC), modulating the physiologic function of TLR9 or TLR10, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. TLR9 or TLR10 antibodies conjugated to toxic or therapeutic agents, such as radioligands or cytosolic toxins, may also be used therapeutically to deliver the toxic or therapeutic agent directly to TLR9- or TLR10-bearing tumor cells.

TLR9 or TLR10 antibodies may be used to suppress the immune system in patients receiving organ transplants or in patients with autoimmune diseases such as arthritis. Healthy immune cells would be targeted by these antibodies leading their death and clearance from the system, thus suppressing the immune system.

TLR9 or TLR10 antibodies may be used as antibody therapy for solid tumors which express this action. Cancer immunotherapy using antibodies provides a novel approach to treating cancers associated with cells that specifically express TLR9 or TLR10. As described above, human toll-like receptors were shown to be expressed on antigen presenting cells, such as monocytes and dendritic cells (WO 01/55386 A1), human colon cancer cell lines (DLD and LoVo), human hepatocellular carcinoma (PLC/PRF/5) and acute myeloid leukemia (KG-1) cells (Yoshioka, et al., J. Int. Med. Res. 29:409-420 (2001)). Table 1 shows that TLR9 was found to be expressed at high levels in the B cell lymphoma cell lines CA-46, RL, GA-10 and HT, moderate levels in promyelomonocytic cell line HL-60 and the acute myeloid leukemia cell line AML-193, and at low levels in acute myeloid leukemia KG-1 cell line. These results demonstrate that TLR9 mRNA is highly expressed in cell lines derived from B cell lymphomas and myeloid leukemias. The results in Table 2 show that levels of mRNA expression were low to moderate in B cell lymphoma tissue (5348, 6879, 6796, 5856, 22601), Hodgkin's disease, acute myeloid leukemia and healthy peripheral blood monocytes (CD-14+). Medium levels of expression were observed in non-cancerous tonsilar lymph nodes, healthy peripheral blood B cells (CD19+ cells), and lung tissue. These findings demonstrate TLR9 mRNA expression in different Non-Hodgkin's B cell lymphoma tissues and cell lines, and indicate that TLR9 may be used as an immunotherapeutic antibody target and a diagnostic marker for certain cell types or disorders (e.g., B-cell lymphomas, T cell lymphomas, myeloid leukemia, Hodgkin's disease). The results in Table 3 show that TLR9 is expressed in the following leukemias and lymphomas: acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, and lymphosarcoma cell leukemia. Thus, targeting cells expressing TLR9 will be useful in treating these diseases. TLR9 is also expressed in solid tumors, such as prostate, breast, colon and squamous cell carcinoma (see Table 4). Thus, targeting TLR9 may be effective in treating these and other non-hematopoietic type tumors (solid tumors). Table 7 shows that TLR10 was found to be expressed at high levels in the B cell lymphoma cell lines CA-46 and RA1, and moderately expressed in the B cell lymphoma cell lines DB, HT, and RL. These results demonstrate that TLR10 mRNA is highly expressed in cell lines derived from B cell lymphomas. The results in Table 8 show that levels of mRNA expression were high in follicular lymphoma tissue (L5856 and L5348), moderate in diffuse large B cell lymphoma tissue (L6879 and L22601), and anaplastic large T cell lymphoma tissue (L5664), and low in acute myeloid leukemia tissue (AML565). These findings demonstrate TLR10 mRNA expression in different Non-Hodgkin's B cell lymphoma tissues and cell lines, and indicate that TLR10 may be used as an immunotherapeutic antibody target and a diagnostic marker for certain cell types or disorders (e.g., B-cell lymphomas, T cell lymphomas, and myeloid leukemia). The results in Table 9 show that TLR10 is expressed in the following leukemias and lymphomas: acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, and lymphosarcoma cell leukemia. Thus, targeting cells expressing TLR10 will be useful in treating these diseases. TLR10 is also expressed in solid tumors, such as prostate, breast, colon and squamous cell carcinoma (see Table 10); therefore, targeting TLR10 may be useful to treat these and other non-hematopoietic type tumors (solid tumors).

Although TLR9 or TLR10 antibody therapy may be useful for all stages of the foregoing cancers, antibody therapy may be particularly appropriate in advanced or metastatic cancers. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with TLR9 or TLR10 antibodies with tumors resistant to chemotherapeutic agents might induce sensitivity and responsiveness to these agents in combination.

Prior to anti-TLR9 or anti-TLR10 immunotargeting, a patient may be evaluated for the presence and level of TLR9 or TLR10 expression by the cancer cells, preferably using immunohistochemical assessments of tumor tissue, quantitative TLR9 or TLR10 imaging, quantitative RT-PCR, or other techniques capable of reliably indicating the presence and degree of TLR9 or TLR10 expression. For example, a blood or biopsy sample may be evaluated by immunohistochemical methods to determine the presence of TLR9- or TLR10-expressing cells or to determine the extent of TLR9 or TLR10 expression on the surface of the cells within the sample. Methods for immunohistochemical analysis of tumor tissues or released fragments of TLR9 or TLR10 in the serum are well known in the art.

Anti-TLR9 or anti-TLR10 antibodies useful in treating cancers include those, which are capable of initiating a potent immune response against the tumor and those, which are capable of direct cytotoxicity. In this regard, anti-TLR9 or anti-TLR10 mAbs may elicit tumor cell lysis by either complement-mediated or ADCC mechanisms, both of which require an intact Fc portion of the immunoglobulin molecule for interaction with effector cell Fc receptor sites or complement proteins. In addition, anti-TLR9 or anti-TLR10 antibodies that exert a direct biological effect on tumor growth are useful in the practice of the invention. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-TLR9 or anti-TLR10 antibody exerts an anti-tumor effect may be evaluated using any number of in vitro assays designed to determine ADCC, ADMMC, complement-mediated cell lysis, and so forth, as is generally known in the art.

The anti-tumor activity of a particular anti-TLR9 or anti-TLR10 antibody, or combination of anti-TLR9 or anti-TLR10 antibody, may be evaluated in vivo using a suitable animal model. For example, xenogenic lymphoma cancer models wherein human lymphoma cells are introduced into immune compromised animals, such as nude or SCID mice. Efficacy may be predicted using assays, which measure inhibition of tumor formation, tumor regression or metastasis, and the like.

It should be noted that the use of murine or other non-human monoclonal antibodies, human/mouse chimeric mAbs may induce moderate to strong immune responses in some patients. In the most severe cases, such an immune response may lead to the extensive formation of immune complexes, which, potentially, can cause renal failure. Accordingly, preferred monoclonal antibodies used in the practice of the therapeutic methods of the invention are those which are either fully human or humanized and which bind specifically to the target TLR9 or TLR10 antigen with high affinity but exhibit low or no antigenicity in the patient.

The method of the invention contemplates the administration of single anti-TLR9 or anti-TLR10 monoclonal antibodies (mAbs) as well as combinations, or “cocktails”, of different mAbs. Two or more monoclonal antibodies that bind to TLR9 or TLR10 may provide an improved effect compared to a single antibody. Alternatively, a combination of an anti-TLR9 or anti-TLR10 antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects. In addition, the administration of anti-TLR9 or anti-TLR10 mAbs may be combined with other therapeutic agents, including but not limited to various chemotherapeutic agents, androgen-blockers, and immune modulators (e.g., IL-2, GM-CSF). The anti-TLR9 or anti-TLR10 mAbs may be administered in their “naked” or unconjugated form, or may have therapeutic agents conjugated to them. Additionally, bispecific antibodies may be used. Such an antibody would have one antigenic binding domain specific for TLR9 or TLR10 and the other antigenic binding domain specific for another antigen (such as CD20 for example). Finally, Fab TLR9 or TLR10 antibodies or fragments of these antibodies (including fragments conjugated to other protein sequences or toxins) may also be used as therapeutic agents.

(1) Anti-TLR9 and Anti-TLR10 Antibodies

Antibodies that specifically bind TLR9 or TLR10 are useful in compositions and methods for immunotargeting cells expressing TLR9 or TLR10 and for diagnosing a disease or disorder wherein cells involved in the disorder express TLR9 or TLR10. Such antibodies include monoclonal and polyclonal antibodies, single chain antibodies, chimeric antibodies, bifunctional/bispecific antibodies, humanized antibodies, human antibodies, and complementary determining region (CDR)-grafted antibodies, including compounds that include CDR and/or antigen-binding sequences, which specifically recognize TLR9 or TLR10. Antibody fragments, including Fab, Fab′, F(ab′)₂, and Fv, are also useful.

The term “specific for” indicates that the variable regions of the antibodies recognize and bind TLR9 or TLR10 exclusively (i.e., able to distinguish TLR9 or TLR10 from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays in which one can determine binding specificity of an anti-TLR9 or anti-TLR10 antibody are well known and routinely practiced in the art. (Chapter 6, Antibodies A Laboratory Manual, Eds. Harlow, et al., Cold Spring Harbor Laboratory; Cold Spring Harbor, N.Y. (1988)).

TLR9 or TLR10 polypeptides can be used to immunize animals to obtain polyclonal and monoclonal antibodies that specifically react with TLR9 or TLR10, respectively. Such antibodies can be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides have been previously described (Merrifield, J. Amer. Chem. Soc. 85, 2149-2154 (1963); Krstenansky, et al., FEBS Lett. 211:10 (1987)). Techniques for preparing polyclonal and monoclonal antibodies as well as hybridomas capable of producing the desired antibody have also been previously disclosed (Campbell, Monoclonal Antibodies Technology: Laboratory Techniques in Biochemistry and Molecular Biology, Elsevier Science Publishers, Amsterdam, The Netherlands (1984); St. Groth, et al., J. Immunol. 35:1-21 (1990); Kohler and Milstein, Nature 256:495-497 (1975)), the trioma technique, the human B-cell hybridoma technique (Kozbor, et al., Immunology Today 4:72 (1983); Cole, et al., in, Monoclonal Antibodies and Cancer Therapy, Alan R. Liss, Inc., pp. 77-96 (1985)).

Any animal capable of producing antibodies can be immunized with a TLR9 or TLR10 peptide or polypeptide. Methods for immunization include subcutaneous or intraperitoneal injection of the polypeptide. The amount of the TLR9 or TLR10 peptide or polypeptide used for immunization depends on the animal that is immunized, antigenicity of the peptide and the site of injection. The TLR9 or TLR10 peptide or polypeptide used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell that produces an antibody with the desired characteristics. These include screening the hybridomas with an ELISA assay, Western blot analysis, or radioimmunoassay (Lutz, et al., Exp. Cell Res. 175:109-124 (1988)). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single chain antibodies can be adapted to produce single chain antibodies to TLR9 or TLR10 (U.S. Pat. No. 4,946,778).

For polyclonal antibodies, antibody-containing antiserum is isolated from the immunized animal and is screened for the presence of antibodies with the desired specificity using one of the above-described procedures.

Because antibodies from rodents tend to elicit strong immune responses against the antibodies when administered to a human, such antibodies may have limited effectiveness in therapeutic methods of the invention. Methods of producing antibodies that do not produce a strong immune response against the administered antibodies are well known in the art. For example, the anti-TLR9 or anti-TLR10 antibody can be a nonhuman primate antibody. Methods of making such antibodies in baboons are disclosed in WO 91/11465 and Losman et al., Int. J. Cancer 46:310-314 (1990). In one embodiment, the anti-TLR9 or anti-TLR10 antibody is a humanized monoclonal antibody. Methods of producing humanized antibodies have been previously described. (U.S. Pat. Nos. 5,997,867 and 5,985,279, Jones et al., Nature 321:522 (1986); Riechmann et al., Nature 332:323(1988); Verhoeyen et al., Science 239:1534-1536 (1988); Carter et al., Proc. Nat'l Acad. Sci. USA 89:4285-4289 (1992); Sandhu, Crit. Rev. Biotech. 12:437-462 (1992); and Singer, et al., J. Immun. 150:2844-2857 (1993)). In another embodiment, the anti-TLR9 or anti-TLR10 antibody is a human monoclonal antibody. Humanized antibodies are produced by transgenic mice that have been engineered to produce human antibodies. Hybridomas derived from such mice will secrete large amounts of human monoclonal antibodies. Methods for obtaining human antibodies from transgenic mice are described in Green, et al., Nature Genet. 7:13-21 (1994), Lonberg, et al., Nature 368:856 (1994), and Taylor, et al., Int. Immun. 6:579 (1994).

The present invention also includes the use of anti-TLR9 or anti-TLR10 antibody fragments. Antibody fragments can be prepared by proteolytic hydrolysis of an antibody or by expression in E. coli of the DNA coding for the fragment. Antibody fragments can be obtained by pepsin or papain digestion of whole antibodies. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods have been previously described (U.S. Pat. Nos. 4,036,945 and 4,331,647, Nisonoff, et al., Arch Biochem. Biophys. 89:230 (1960); Porter, Biochem. J. 73:119 (1959), Edelman, et al., Meth. Enzymol. 1:422 (1967)). Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V_{H} and V_{L} chains, which can be noncovalent (Inbar et al., Proc. Nat'l Acad. Sci. USA 69:2659 (1972)). Alternatively, the variable chains can be linked by an intermolecular disulfide bond or cross-linked by chemicals such as glutaraldehyde.

In one embodiment, the Fv fragments comprise V_{H} and V_{L} chains that are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing sFvs have been previously described (U.S. Pat. No.4,946,778, Whitlow, et al., Methods: A Companion to Methods in Enzymology 2:97 (1991), Bird, et al., Science 242:423 (1988), Pack, et al., Bio/Technology 11:1271 (1993)).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick, et al., Methods: A Companion to Methods in Enymology 2:106 (1991); Courtenay-Luck, pp. 166-179 in, Monoclonal Antibodies Production, Engineering and Clinical Applications, Eds. Ritter et al., Cambridge University Press (1995); Ward, et al., pp. 137-185 in, Monoclonal Antibodies Principles and Applications, Eds. Birch et al., Wiley-Liss, Inc. (1995)).

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling have been previously disclosed (Sternberger, et al., J. Histochem. Cytochem. 18:315 (1970); Bayer, et al., Meth. Enzym. 62:308 (1979); Engval, et al., Immunol. 109:129 (1972); Goding, J. Immunol. Meth. 13:215 (1976)).

The labeled antibodies can be used for in vitro, in vivo, and in situ assays to identify cells or tissues in which TLR9 or TLR10 is expressed. Furthermore, the labeled antibodies can be used to identify the presence of secreted TLR9 or TLR10 in a biological sample, such as a blood, urine, and saliva samples.

(2) Anti-TLR9 and Anti-TLR10 Antibody Conjugates

The present invention contemplates the use of “naked” anti-TLR9 or anti-TLR10 antibodies, as well as the use of immunoconjugates. Immnunoconjugates can be prepared by indirectly conjugating a therapeutic agent such as a cytotoxic agent to an antibody component. Toxic moieties include, for example, plant toxins, such as abrin, ricin, modeccin, viscumin, pokeweed anti-viral protein, saporin, gelonin, momoridin, trichosanthin, barley toxin; bacterial toxins, such as Diptheria toxin, Pseudomonas endotoxin and exotoxin, Staphylococcal enterotoxin A; fungal toxins, such as α-sarcin, restrictocin; cytotoxic RNases, such as extracellular pancreatic RNases; DNase I (Pastan, et al., Cell 47:641 (1986); Goldenberg, Cancer Journal for Clinicians 44:43 (1994)), calicheamicin, and radioisotopes, such as ³²P, ⁶⁷Cu, ⁷⁷As, ¹⁰⁵Rh, ¹⁰⁹Pd, ¹¹¹Ag, ¹²¹Sn, ¹³¹I, ¹⁶⁶Ho, ¹⁷⁷Lu, ¹⁸⁶Re, ¹⁸⁸Re, ⁹⁴¹Ir, ¹⁹⁹Au (Illidge, T. M. & Brock, S., Curr Pharm. Design 6: 1399 (2000)). In humans, clinical trials are underway utilizing a yttrium-90 conjugated anti-CD20 antibody for B cell lymphomas (Cancer Chemother Pharmacol 48(Suppl 1):S91-S95 (2001)).

General techniques have been previously described (U.S. Pat. Nos. 6,306,393 and 5,057,313, Shih, et al., Int. J. Cancer 41:832-839 (1988); Shih, et al., Int. J. Cancer 46:1101-1106 (1990)). The general method involves reacting an antibody component having an oxidized carbohydrates portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an immunoconjugate with an aminodextran carrier typically begins with a dextran polymer, advantageously a dextran of average molecular weight of about 10,000-100,000. The dextran is reacted with an oxidizing agent to affect a controlled oxidation of a portion of its carbohydrate rings to generate aldehyde groups. The oxidation is conveniently effected with glycolytic chemical reagents such as NaIO₄, according to conventional procedures. The oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to ensure substantially complete conversion of the aldehyde functions to Schiff base groups. A reducing agent, such as NaBH₄, NaBH₃ CN or the like, is used to effect reductive stabilization of the resultant Schiff base intermediate. The resultant adduct can be purified by passage through a conventional sizing column or ultrafiltration membrane to remove cross-linked dextrans. Other conventional methods of derivatizing a dextran to introduce amine functions can also be used, e.g., reaction with cyanogen bromide, followed by reaction with a diamine.

The amninodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct. Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well-known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier should have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylygine, polyglutamic acid, polyaspartic acid, co-polymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaIO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate-portion of an antibody component. Carboxyls on the polypeptide carrier can, if necessary, be converted to amines by, e.g., activation with DCC and reaction with an excess of a diamine.

The final immunoconjugate is purified using conventional techniques, such as sizing chromatography on Sephacryl S-300 or affinity chromatography using one or more TLR9 or TLR10 epitopes.

Alternatively, immunoconjugates can be prepared by directly conjugating an antibody component with a therapeutic agent. The general procedure is analogous to the indirect method of conjugation except that a therapeutic agent is directly attached to an oxidized antibody component. It will be appreciated that other therapeutic agents can be substituted for the chelators described herein. Those of skill in the art will be able to devise conjugation schemes without undue experimentation.

As a further illustration, a therapeutic agent can be attached at the hinge region of a reduced-antibody component via disulfide bond formation. For example, the tetanus toxoid peptides can be constructed with a single cysteine residue that is used to attach the peptide to an antibody component. As an alternative, such peptides can be attached to the antibody component using a heterobifunctional cross-linker, such as N-succinyl 3-(2-pyridyldithio)proprionate (SPDP) (Yu, et al., Int. J. Cancer 56:244 (1994)). General techniques for such conjugation have been previously described (Wong, Chemistry of Protein Conjugation and Cross-linking, CRC Press (1991); Upeslacis, et al., pp. 187-230 in, Monoclonal Antibodies Principles and Applications, Eds. Birch et al., Wiley-Liss, Inc. (1995); Price, pp. 60-84 in, Monoclonal Antibodies: Production, Engineering and Clinical Applications Eds. Ritter, et al., Cambridge University Press (1995)).

As described above, carbohydrate moieties in the Fc region of an antibody can be used to conjugate a therapeutic agent. However, the Fc region may be absent if an antibody fragment is used as the antibody component of the immunoconjugate. Nevertheless, it is possible to introduce a carbohydrate, moiety into the light chain variable region of an antibody or antibody fragment (Leung, et al., J. Immunol. 154:5919-5926 (1995); U.S. Pat. No. 5,443,953). The engineered carbohydrate moiety is then used to attach a therapeutic agent.

In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

(3) Anti-TLR9 and Anti-TLR10 Antibody Fusion Proteins

When the therapeutic agent to be conjugated to the antibody is a protein, the present invention contemplates the use of fusion proteins comprising one or more anti-TLR9 or anti-TLR10 antibody moieties and an immunomodulator or toxin moiety. Methods of making antibody fusion proteins have been previously described (U.S. Pat. No. 6,306,393). Antibody fusion proteins comprising an interleukin-2 moiety have also been previously disclosed (Boleti, et al., Ann. Oncol. 6:945 (1995), Nicolet, et al., Cancer Gene Ther. 2:161 (1995), Becker, et al., Proc. Nat'l Acad. Sci. USA 93:7826 (1996), Hank, et al., Clin. Cancer Res. 2:1951 (1996), Hu, et al., Cancer Res. 56:4998 (1996)). In addition, Yang, et al., Hum. Antibodies Hybridomas 6:129 (1995), describe a fusion protein that includes an F(ab′)₂ fragment and a tumor necrosis factor alpha moiety.

Methods of making antibody-toxin fusion proteins in which a recombinant molecule comprises one or more antibody components and a toxin or chemotherapeutic agent also are known to those, of skill in the art. For example, antibody-Pseudomonas exotoxin A fusion proteins have been described (Chaudhary, et al., Nature 339:394 (1989), Brinkmann, et al., Proc. Nat'l Acad. Sci. USA 88:8616 (1991), Batra, et al., Proc. Natl. Acad. Sci. USA 89:5867 (1992), Friedman, et al., J. Immunol. 150:3054 (1993), Wels, et al., Int. J. Can. 60:137 (1995), Fominaya et al., J. Biol. Chem. 271:10560 (1996), Kuan, et al., Biochemistry 35:2872 (1996), Schmidt, et al., Int. J Can. 65:538 (1996)). Antibody-toxin fusion proteins containing a diphtheria toxin moiety have been described (Kreitman, et al., Leukemia 7:553 (1993), Nicholls, et al., J. Biol. Chem. 268:5302 (1993), Thompson, et al., J. Biol. Chem. 270:28037 (1995), and Vallera, et al., Blood 88:2342 (1996). Deonarain et al. (Tumor Targeting 1:177 (1995)), have described an antibody-toxin fusion protein having an RNase moiety, while Linardou, et al. (Cell Biophys. 24-25:243 (1994)), produced an antibody-toxin fusion protein comprising a DNase I component. Gelonin and Staphylococcal enterotoxin-A have been used as the toxin moieties in antibody-toxin fusion proteins (Wang, et al., Abstracts of the 209th ACS National Meeting, Anaheim, Calif., Apr. 2-6, 1995, Part 1, BIOT005; Dohlsten, et al., Proc. Nat'l Acad. Sci. USA 91:8945 (1994)).

Diseases Amenable to Anti-TLR9 or Anti-TLR10 Immunotargeting

In one aspect, the present invention provides reagents and methods useful for treating diseases and conditions wherein cells associated with the disease or disorder express TLR9 or TLR10. These diseases can include cancers, and other hyperproliferative conditions, such as hyperplasia, psoriasis, contact dermatitis, immunological disorders, and infertility. Whether the cells associated with a disease or condition express TLR9 or TLR10 can be determined using the diagnostic methods described herein.

Comparisons of TLR9 or TLR10 mRNA and protein expression levels between diseased cells, tissue or fluid (blood, lymphatic fluid, etc.) and corresponding normal samples are made to determine if the patient will be responsive to TLR9 or TLR10 immunotherapy. Methods for detecting and quantifying the expression of TLR9 or TLR10 mRNA or protein use standard nucleic acid and protein detection and quantitation techniques that are well known in the art and are described in Sambrook, et al., Molecular Cloning: A Laboratory Manual, Cold Spring Harbor Laboratory, New York (1989) or Ausubel, et al., Current Protocols in Molecular Biology, John Wiley & Sons, New York, N.Y. (1989), both of which are incorporated herein by reference in their entirety. Standard methods for the detection and quantification of TLR9 or TLR10 mRNA include in situ hybridization using labeled TLR9 or TLR10 riboprobes (Gemou-Engesaeth, et al., Pediatrics 109: E24-E32 (2002)); Northern blot and related techniques using TLR9 or TLR10 polynucleotide probes (Kunzli, et al., Cancer 94: 228 (2002)), RT-PCR analysis using TLR9- or TLR10-specific primers (Angchaiskisiri, et al., Blood 99:130 (2002)), and other amplification detection methods, such as branched chain DNA solution hybridization assay (Jardi, et al., J. Viral Hepat. 8:465-471 (2001)), transcription-mediated amplification (Kimura, et al., J. Clin. Microbiol. 40:439-445 (2002)), microarray products, such as oligos, cDNAs, and monoclonal antibodies, and real-time PCR (Simpson, et al., Molec. Vision, 6:178-183 (2000)). Standard methods for the detection and quantification of TLR9 or TLR10 protein include western blot analysis (Sambrook, et al., Molecular Cloning: A Laboratory Manual, Cold Spring Harbor Laboratory, New York (1989), Ausubel, et al., Current Protocols in Molecular Biology, John Wiley & Sons, New York, N.Y. (1989)), immunocytochemistry (Racila, et al., Proc. Natl. Acad. Sci. USA 95:4589-4594 (1998)), and a variety of immunoassays, including enzyme-linked immunosorbant assay (ELISA), radioimmunoassay (RIA), and specific enzyme immunoassay (EIA) (Sambrook, et al., Molecular Cloning: A Laboratory Manual, Cold Spring Harbor Laboratory, New York (1989), Ausubel, et al., Current Protocols in Molecular Biology, John Wiley & Sons, New York, N.Y. (1989)). Peripheral blood cells can also be analyzed for TLR9 or TLR10 expression using flow cytometry using, for example, immunomagnetic beads specific for TLR9 or TLR10 (Racila, et al., Proc. Natl. Acad. Sci. USA 95:4589-4594 (1998)) or biotinylated TLR9 or TLR10 antibodies (Soltys, et al., J. Immunol. 168:1903 (2002)). Tumor aggressiveness can be gauged by determining the levels of TLR9 or TLR10 protein or mRNA in tumor cells compared to the corresponding normal cells (Orlandi, et al., Cancer Res. 62:567 (2002)). In one embodiment, the disease or disorder is a cancer. Cancer, a leading cause of death in the United States, causes over a half-million deaths annually. As the population ages, the numbers of deaths due to cancer are expected to rise significantly. Cancer is a general term and encompasses various types of malignant neoplasms, most of which invade surrounding tissues, may metastasize to several sites, and are likely to recur after attempted removal and to cause death of the patient unless adequately treated. Cancer can develop in any tissue of any organ at any age. Once a cancer diagnosis is made, treatment decisions are paramount. Successful therapy focuses on the primary tumor and its metastases. Various types of cancer treatments have been developed to improve the survival and quality of life of cancer patients. Advances in cancer treatment include new cytotoxic agents and new surgical and radiotherapy techniques. However, many of these treatments have substantial emotional and physical drawbacks. Furthermore, treatment failure remains a common occurrence. Such shortcomings have driven cancer researchers and caregivers to develop new and effective ways of treating cancer.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of tissue cells in which the multiplication of the cells is uncontrolled and progressive. Some such growths are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is also called “anaplasia.”

Neoplasms treatable by the present invention also include solid phase tumors/malignancies, i.e., carcinomas, locally advanced tumors and human soft tissue sarcomas. Carcinomas include those malignant neoplasms derived from epithelial cells that infiltrate (invade) the surrounding tissues and give rise to metastastic cancers, including lymphatic metastases. Adenocarcinomas are carcinomas derived from glandular tissue, or which form recognizable glandular structures. Another broad category or cancers includes sarcomas, which are tumors whose cells are embedded in a fibrillar or homogeneous substance like embryonic connective tissue. As seen in Tables 4 and 10, TLR9 (Table 4) and TLR10 (Table 10) ae expressed in breast, colon, prostate, and squamous cell carcinoma. It is likely that other epithelial and squamous cell cancers will express TLR9 and/or TLR10 as well. In addition, other solid phase tumors may express TLR9 and/or TLR10. The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells that may be amenable to treatment according to the invention include, for example, acute lymphocytic leukemia, acute nonlymphocytic leukemia, chronic lymphocytic leukemia, chronic myelocytic leukemia, cutaneous T-cell lymphoma, hairy cell leukemia, acute myeloid leukemia, erythroleukemia, chronic myeloid (granulocytic) leukemia, Hodgkin's disease, and non-Hodgkin's lymphoma, gastrointestinal cancers including esophageal cancer, stomach cancer, colon cancer, colorectal cancer, polyps associated with colorectal neoplasms, pancreatic cancer and gallbladder cancer, cancer of the adrenal cortex, ACTH-producing tumor, bladder cancer, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumors in the ovarian follicle, vaginal cancer, cancer of the vulva, and cervical cancer; breast cancer (small cell and ductal), penile cancer, prostate cancer, retinoblastoma, testicular cancer, thyroid cancer, trophoblastic neoplasms, and Wilms' tumor.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any cancer derived from any organ system. As demonstrated in the Examples, TLR9 and TLR10 are highly expressed in B-cell related disorders. Leukemias can result from uncontrolled B cell proliferation initially within the bone marrow before disseminating to the peripheral blood, spleen, lymph nodes and finally to other tissues. Uncontrolled B cell proliferation also may result in the development of lymphomas that arise within the lymph nodes and then spread to the blood and bone marrow. Immunotargeting of TLR9 or TLR10 is used in treating B cell malignancies, leukeminas, lymphomas and myelomas including but not limited to multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma (NHL), B cell chronic lymphocytic leukemia (CLL), acute lymphoblastic leukemia, hairy cell leukemia (HCL), acute myelogenous leukemia, acute myelomonocytic leukemia, chronic myelogenous leukemia, lymphosarcoma cell leukemia, B cell large cell lymphoma, malignant lymphoma, splenic marginal zone lymphoma, diffuse large B cell lymphoma, prolymphocytic leukemia (PLL), lymphoplasma cytoid lymphoma, mantle cell lymphoma, mucosa-associated lymphoid tissue (MALT) lymphoma, primary thyroid lymphoma, intravascular malignant lymphomatosis, splenic lymphoma, Hodgkin's Disease, and intragraft angiotropic large-cell lymphoma. Expression of TLR9 or TLR10 has also been demonstrated in Examples 1-4 in myeloid leukemia cell lines and tissue, T cell leukemia cell lines and T cell lymphoma tissues and may be treated with TLR9 or TLR10 antibodies. Other diseases that may be treated by the methods of the present invention include multicentric Castleman's disease, primary amyloidosis, Franklin's disease, Seligmann's disease, primary effusion lymphoma, post-transplant lymphoproliferative disease (PTLD) [associated with EBV infection.], paraneoplastic pemphigus, chronic lymphoproliferative disorders, X-linked lymphoproliferative syndrome (XLP), acquired angioedema, angioimmunoblastic lymphadenopathy with dysproteinemia, Herman's syndrome, post-splenectomy syndrome, congenital dyserythropoietic anemia type III, lymphoma-associated hemophagocytic syndrome (LAHS), necrotizing ulcerative stomatitis, Kikuchi's disease, lymphomatoid granulomatosis, Richter's syndrome, polycythemic vera (PV), Gaucher's disease, Gougerot-Sjogren syndrome, Kaposi's sarcoma, cerebral lymphoplasmocytic proliferation (Bind and Neel syndrome), X-linked lymphoproliferative disorders, pathogen associated disorders such as mononucleosis (Epstein Barr Virus), lymphoplasma cellular disorders, post-transplantational plasma cell dyscrasias, and Good's syndrome.

Autoimmune diseases can be associated with hyperactive B cell activity that results in autoantibody production. Inhibition of the development of autoantibody-producing cells or proliferation of such cells may be therapeutically effective in decreasing the levels of autoantibodies in autoimmune diseases including but not limited to systemic lupus erythematosus, Crohn's Disease, graft-verses-host disease, Graves' disease, myasthenia gravis, autoimmune hemolytic anemia, autoimmune thrombocytopenia, asthma, cryoglubulinemia, primary biliary sclerosis, pernicious anemia, Waldenstrom macroglobulinemia, hyperviscosity syndrome, macroglobulinemia, cold agglutinin disease, monoclonal gammopathy of undetermined origin, anetoderma and POEMS syndrome (polyneuropathy, organomegaly, endocrinopathy, M component, skin changes), connective tissue disease, multiple sclerosis, cystic fibrosis, rheumatoid arthritis, autoimmune pulmonary inflammation, psoriasis, Guillain-Barre syndrome, autoimmune thyroiditis, insulin dependent diabetes mellitis, autoimmune inflammatory eye disease, Goodpasture's disease, Rasmussen's encephalitis, dermatitis herpetiformis, thyoma, autoimmune polyglandular syndrome type 1, primary and secondary membranous nephropathy, cancer-associated retinopathy, autoimmune hepatitis type 1, mixed cryoglobulinemia with renal involvement, cystoid macular edema, endometriosis, IgM polyneuropathy (including Hyper IgM syndrome), demyelinating diseases, angiomatosis, and monoclonal gammopathy. As shown in Tables 5 and 11, IHC analysis showed TLR9 (Table 5) and TLR10 (Table 11) expression on tissues from the following autoimmune disorders: systemic lupus erythematosus, Hasimoto thyroiditis, Sjörgen's syndrome, and pericarditis lupus; thus targeting TLR9 and TLR10 may be useful therapies for these and other autoimmune disorders.

Immunotargeting of TLR9 or TLR10 may also be useful in the treatment of allergic reactions and conditions e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, allergic gastroenteropathy, inflammatory bowel disorder (IBD), and contact allergies, such as asthma (particularly allergic asthma), or other respiratory problems.

Immunotargeting TLR9 or TLR10 may also be useful in the management or prevention of transplant rejection in patients in need of transplants such as stem cells, tissue or organ transplant. Thus, one aspect of the invention may find therapeutic utility in various diseases (such as those usually treated with transplantation, including without limitation, aplastic aneria and paroxysmal nocturnal hemoglobinuria) as wells in repopulating the stem cell compartment post irridiation/chemotherapy, either in vivo or ex vivo (i.e. in conjunction with bone marrow transplantation or with peripheral progenitor cell transplantation (homologous or heterologous) as normal cells or genetically manipulated for gene therapy. As shown in Tables 6 and 12, IHC analysis showed TLR9 (Table 6) and TLR10 (Table 12) are expressed in rejected heart, liver and kidney tissue after transplantation as opposed to normal tissue. Thus, targeting of TLR9 and TLR10 may be useful to prevent and/or reduce tissue rejection after transplantation.

Immunotargeting of TLR9 or TLR10 may also be possible to modulate immune responses, in a number of ways. Down regulation may be in the form of inhibiting or blocking an immune response already in progress or may involve preventing the induction of an immune response. Down regulating or preventing one or more antigen functions (including without limitation B lymphocyte antigen functions (such as, for example, TLR9 or TLR10)), e.g., modulating or preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells (i.e. cytotoxic T lymphocytes), followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of therapeutic compositions of the invention on the development of that disease.

Administration

The anti-TLR9 or anti-TLR10 monoclonal antibodies used in the practice of a method of the invention may be formulated into pharmaceutical compositions comprising a carrier suitable for the desired delivery method. Suitable carriers include any material which when combined with the anti-TLR9 or anti-TLR10 antibodies retain the anti-tumor function of the antibody and are nonreactive with the subject's immune systems. Examples include, but are not limited to, any of a number of standard pharmaceutical carriers such as sterile phosphate buffered saline solutions, bacteriostatic water, and the like.

The anti-TLR9 or anti-TLR10 antibody formulations may be administered via any route capable of delivering the antibodies to the tumor site. Potentially effective routes of administration include, but are not limited to, intravenous, intraperitoneal, intramuscular, intratumor, intradermal, and the like. The preferred route of administration is by intravenous injection. A preferred formulation for intravenous injection comprises anti-TLR9 or anti-TLR10 mAbs in a solution of preserved bacteriostatic water, sterile unpreserved water, and/or diluted in polyvinylchloride or polyethylene bags containing 0.9% sterile sodium chloride for Injection, USP. The anti-TLR9 or anti-TLR10 mAb preparation may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the anti-TLR9 or anti-TLR10 antibody preparation via an acceptable route of administration such as intravenous injection (IV), typically at a dose in the range of about 0.1 to about 10 mg/kg body weight; however other exemplary doses in the range of 0.01 mg/kg to about 100 mg/kg are also contemplated. Doses in the range of 10-500 mg mAb per week may be effective and well tolerated. Rituximab (Rituxan®), a chimeric CD20 antibody used to treat B-cell lymphoma, non-Hodgkin's lymphoma, and relapsed indolent lymphoma, is typically administered at 375 mg/m² by IV infusion once a week for 4 to 8 doses. Sometimes a second course is necessary, but no more than 2 courses are allowed. An effective dosage range for Rituxan® would be 50 to 500 mg/m² (Maloney, et al., Blood 84: 2457-2466 (1994); Davis, et al., J. Clin. Oncol. 18: 3135-3143 (2000)). Based on clinical experience with Trastuzumab (Herceptin®), a humanized monoclonal antibody used to treat HER2 (human epidermal growth factor 2)-positive metastatic breast cancer (Slamon, et al., Mol Cell Biol. 9: 1165 (1989)), an initial loading dose of approximately 4 mg/kg patient body weight IV followed by weekly doses of about 2 mg/kg IV of the anti-TLR9 or anti-TLR10 mAb preparation may represent an acceptable dosing regimen (Slamon, et al., N. Engl. J. Med. 344: 783(2001)). Preferably, the initial loading dose is administered as a 90-minute or longer infusion. The periodic maintenance dose may be administered as a 30 minute or longer infusion, provided the initial dose was well tolerated. However, as one of skill in the art will understand, various factors will influence the ideal dose regimen in a particular case. Such factors may include, for example, the binding affinity and half life of the mAb or mAbs used, the degree of TLR9 or TLR10 overexpression in the patient, the extent of circulating shed TLR9 or TLR10 antigen, the desired steady-state antibody concentration level, frequency of treatment, and the influence of chemotherapeutic agents used in combination with the treatment method of the invention.

Treatment can also involve anti-TLR9 or anti-TLR10 antibodies conjugated to radioisotopes. Studies using radiolabeled-anticarcinoembryonic antigen (anti-CEA) monoclonal antibodies, provide a dosage guideline for tumor regression of 2-3 infusions of 30-80 mCi/m² (Behr, et al. Clin, Cancer Res. 5(10 Suppl.): 3232s-3242s (1999), Juweid, et al., J. Nucl. Med. 39:34-42 (1998)).

Alternatively, dendritic cells transfected with mRNA encoding TLR9 or TLR10 can be used as a vaccine to stimulate T-cell mediated anti-tumor responses. Studies with dendritic cells transfected with prostate-specific antigen mRNA suggest 3 cycles of intravenous administration of 1×10^{7-5×10}⁷ cells for 2-6 weeks concomitant with an intradermal injection of 10⁷ cells may provide a suitable dosage regimen (Heiser, et al., J. Clin. Invest. 109:409-417 (2002); Hadzantonis and O'Neill, Cancer Biother. Radiopharm. 1:11-22(1999)). Other exemplary doses of between 1×10⁵ to 1×10⁹ or 1×10⁶ to 1×10⁸ cells are also contemplated.

Naked DNA vaccines using plasmids encoding TLR9 or TLR10 can induce an immunologic anti-tumor response. Patent application WO200188200-A2 teaches the use of naked DNA vaccines by particle bombardment technique where Ly-9 (a member of the immunoglobulin (Ig) superfamily) DNA-coated microprojectiles at high velocity are used to pierce cell membranes and enter cells without killing the cells. Administration of naked DNA by direct injection into the skin and muscle is not associated with limitations encountered using viral vectors, such as the development of adverse immune reactions and risk of insertional mutagenesis (Hengge, et al., J. Invest. Dermatol. 116:979 (2001)). Studies have shown that direct injection of exogenous cDNA into muscle tissue results in a strong immune response and protective immunity (Ilan, Curr. Opin. Mol. Ther. 1:116-120 (1999)). Physical (gene gun, electroporation) and chemical (cationic lipid or polymer) approaches have been developed to enhance efficiency and target cell specificity of gene transfer by plasmid DNA (Nishikawa and Huang, Hum. Gene Ther. 12:861-870 (2001)). Plasmid DNA can also be administered to the lungs by aerosol delivery (Densmore, et al., Mol. Ther. 1:180-188 (2000)). Gene therapy by direct injection of naked or lipid-coated plasmid DNA is envisioned for the prevention, treatment, and cure of diseases such as cancer, acquired immunodeficiency syndrome, cystic fibrosis, cerebrovascular disease, and hypertension (Prazeres, et al., Trends Biotechnol. 17:169-174 (1999); Weihi, et al., Neurosurgery 44:239-252 (1999)). HIV-1 DNA vaccine dose-escalating studies indicate administration of 30-300 μg/dose as a suitable therapy (Weber, et al., Eur. J. Clin. Microbiol. Infect. Dis. 20: 800). Naked DNA injected intracerebrally into the mouse brain was shown to provide expression of a reporter protein, wherein expression was dose-dependent and maximal for 150 μg DNA injected (Schwartz, et al., Gene Ther. 3:405-411 (1996)) Gene expression in mice after intramuscular injection of nanospheres containing 1 microgram of beta-galactosidase plasmid was greater and more prolonged than was observed after an injection with an equal amount of naked DNA or DNA complexed with Lipofectamine (Truong, et al., Hum. Gene Ther. 9:1709-1717 (1998)). In a study of plasmid-mediated gene transfer into skeletal muscle as a means of providing a therapeutic source of insulin, wherein four plasmid constructs comprising a mouse furin cDNA transgene and rat proinsulin cDNA were injected into the calf muscles of male Balb/c mice, the optimal dose for most constructs was 100 micrograms plasmid DNA (Kon, et al. J. Gene Med. 1:186-194 (1999)). Other exemplary doses of 1-1000 μg/dose or 10-500 μg/dose are also contemplated.

Optimally, patients should be evaluated for the level of circulating shed TLR9 or TLR10 antigen in serum in order to assist in the determination of the most effective dosing regimen and related factors. Such evaluations may also be used for monitoring purposes throughout therapy, and may be useful to gauge therapeutic success in combination with evaluating other parameters.

(1) TLR9 and TLR10 Targeting Compositions

Compositions for targeting TLR9- or TLR10-expressing cells are within the scope of the present invention. Pharmaceutical compositions comprising antibodies are described in detail in, for example, U.S. Pat. No. 6,171,586, to Lam et al., issued Jan. 9, 2001. Such compositions comprise a therapeutically or prophylactically effective amount an antibody, or a fragment, variant, derivative or fusion thereof as described herein, in admixture with a pharmaceutically acceptable agent. Typically, the TLR9 or TLR10 immunotargeting agent will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, Ed. A. R. Gennaro, Mack Publishing Company, (1990)).

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate of in vivo release, and rate of in vivo clearance of the TLR9 or TLR10 immunotargeting agent.

The primary vehicle or carrier in a pharmaceutical composition may be either aqueous or non-aqueous in nature. For example, a suitable vehicle or carrier may be water for injection, physiological saline solution or artificial-cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, TLR9 or TLR10 immunotargeting agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for delivery through the digestive tract, such as orally. The preparation of such pharmaceutically acceptable compositions is within the skill of the art. The formulation components are present in concentrations that are acceptable to the site of administration. For example, buffers are used to maintain the composition at physiological pH or at slightly lower pH, typically within a pH range of from about 5 to about 8. When parenteral administration is contemplated, the therapeutic compositions for use in this invention may be in the form of a pyrogen-free, parenterally acceptable aqueous solution comprising the TLR9 or TLR10 immunotargeting agent in a pharmaceutically acceptable vehicle. A particularly suitable vehicle for parenteral injection is sterile distilled water in which a TLR9 or TLR10 immunotargeting agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

In another embodiment, a pharmaceutical composition may be formulated for inhalation. For example, a TLR9 or TLR10 immunotargeting agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, TLR9 or TLR10 immunotargeting agents that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the TLR9 or TLR10 immunotargeting agent may be dissolved or suspended in suitable liquids, such as fatty oils, liquid, or liquid polyethylene glycol with or without stabilizers.

Another pharmaceutical composition may involve an effective quantity of TLR9 or TLR10 immunotargeting agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving TLR9 or TLR10 immunotargeting agents in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 (1983)), poly (2-hydroxyethyl-methacrylate) (Langer et al., J Biomed Mater Res, 15:167-277, (1981)) and (Langer et al., Chem Tech, 12:98-105(1982)), ethylene vinyl acetate (Langer et al., supra) or poly-D (−)-3-hydroxybutyric acid (EP 133,988). Sustained-release compositions also include liposomes, which can be prepared by any of several methods known in the art. See e.g., Epstein, et al., Proc Natl Acad Sci (USA), 82:3688-3692 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried TLR9 or TLR10 immunotargeting agent and a second container having an aqueous formulation. Also included within the scope of this invention are kits containing single and multi-chambered pre-filled syringes (e.g., liquid syringes and lyosyringes).

(2) Dosage

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which TLR9 or TLR10 immunotargeting agent is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 0.01 mg/kg to 1 g/kg; or 1 mg/kg up to about 100 mg/kg or 5 mg/kg up to about 100 mg/kg. In other embodiments, the dosage may range from 10 mCi to 100 mCi per dose for radioimmunotherapy, from about 1×10⁷ to 5×10⁷ cells or 1×10⁵to 1×10⁹ cells or 1×10⁶ to 1×10⁸ cells per injection or infusion, or from 30 μg to 300 μg naked DNA per dose or 1-1000 μg/dose or 10-500 μg/dose, depending on the factors listed above.

For any compound, the therapeutically effective dose can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the TLR9 or TLR10 immunotargeting agent in the formulation used. Typically, a composition is administered until a dosage is reached that achieves the desired effect. The composition may therefore be administered as a single dose or as multiple doses (at the same or different concentrations/dosages) over time, or as a continuous infusion. Further refinement of the appropriate dosage is routinely made. Appropriate dosages may be ascertained through use of appropriate dose-response data.

(3) Routes of Administration

The route of administration of the pharmaceutical composition is in accord with known methods, e.g. orally, through injection by intravenous, intraperitoneal, intracerebral (intra-parenchymal), intracerebroventricular, intramuscular, intra-ocular, intra-arterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the TLR9 or TLR10 immunotargeting agent has been absorbed or encapsulated. Where an implantation device is used, the device may be implanted into any suitable tissue or organ, and delivery of the TLR9 or TLR10 immunotargeting agent may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

In other cases, a TLR9 or TLR10 immunotargeting agent can be delivered by implanting certain cells that have been genetically engineered to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Combination Therapy

TLR9 or TLR10 targeting agents of the invention can be utilized in combination with other therapeutic agents. These other therapeutics include, for example radiation treatment, chemotherapeutic agents, as well as other growth factors.

In one embodiment, anti-TLR9 or anti-TLR10 antibody is used as a radiosensitizer. In such embodiments, the anti-TLR9 or anti-TLR10 antibody is conjugated to a radiosensitizing agent. The term “radiosensitizer,” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells.

The terms “electromagnetic radiation” and “radiation” as used herein include, but are not limited to, radiation having the wavelength of 10⁻²⁰ to 100 meters. Preferred embodiments of the present invention employ the electromagnetic radiation of: gamma-radiation (10⁻²⁰ to 10⁻¹³ m), X-ray radiation (10⁻¹² to 10⁻⁹ m), ultraviolet light (10 nm to 400 nm), visible light (400 nm to 700 nm), infrared radiation (700 nm to 1.0 mm), and microwave radiation (1 mm to 30 cm).

Radiosensitizers are known to increase the sensitivity of cancerous cells to the toxic effects of electromagnetic radiation. Many cancer treatment protocols currently employ radiosensitizers activated by the electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FUdR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin(r), benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Chemotherapy treatment can employ anti-neoplastic agents including, for example, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; ppipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Combination therapy with growth factors can include cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF0, TNF1, TNF2, G-CSF, Meg-CSF, GM-CSF, thrombopoietin, stem cell factor, and erythropoietin. Other compositions can include known angiopoietins, for example, vascular endothelial growth factor (VEGF). Growth factors include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2, cytokine-induced neutrophil chemotactic factor 2, endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor 1, glial cell line-derived neutrophic factor receptor 2, growth related protein, growth related protein, growth related protein, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, platelet derived growth factor receptor, pre-B cell growth stimulating factor, stem cell factor, stem cell factor receptor, transforming growth factor, transforming growth factor, transforming growth factor 1, transforming growth factor 1.2, transforming growth factor 2, transforming growth factor 3, transforming growth factor 5, latent transforming growth factor 1, transforming growth factor binding protein I, transforming growth factor binding protein II, transforming growth factor binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Diagnostic Uses of TLR9 or TLR10

(1) Assays for Determining TLR9 or TLR10 Expression Status

Determining the status of TLR9 or TLR10 expression patterns in an individual may be used to diagnose cancer and may provide prognostic information useful in defining appropriate therapeutic options. Similarly, the expression status of TLR9 or TLR10 may provide information useful for predicting susceptibility to particular disease stages, progression, and/or tumor aggressiveness. The invention provides methods and assays for determining TLR9 or TLR10 expression status and diagnosing cancers that express TLR9 or TLR10.

In one aspect, the invention provides assays useful in determining the presence of cancer in an individual, comprising detecting a significant increase in TLR9 or TLR10 mRNA or protein expression in a test cell or tissue or fluid sample relative to expression levels in the corresponding normal cell or tissue. In one embodiment, the presence of TLR9 or TLR10 mRNA is evaluated in tissue samples of a lymphoma. The presence of significant TLR9 or TLR10 expression may be useful to indicate whether the lymphoma is susceptible to TLR9 or TLR10 immunotargeting. In a related embodiment, TLR9 or TLR10 expression status may be determined at the protein level rather than at the nucleic acid level. For example, such a method or assay would comprise determining the level of TLR9 or TLR10 expressed by cells in a test tissue sample and comparing the level so determined to the level of TLR9 or TLR10 expressed in a corresponding normal sample. In one embodiment, the presence of TLR9 or TLR10 is evaluated, for example, using immunohistochemical methods. TLR9 or TLR10 antibodies capable of detecting TLR9 or TLR10 expression may be used in a variety of assay formats well known in the art for this purpose.

Peripheral blood may be conveniently assayed for the presence of cancer cells, including lymphomas and leukemias, using RT-PCR to detect TLR9 or TLR10 expression. The presence of RT-PCR amplifiable TLR9 or TLR10 mRNA provides an indication of the presence of one of these types of cancer. A sensitive assay for detecting and characterizing carcinoma cells in blood may be used (Racila, et al., Proc. Natl. Acad. Sci. USA 95: 4589-4594 (1998)). This assay combines immunomagnetic enrichment with multiparameter flow cytometric and immunohistochemical analyses, and is highly sensitive for the detection of cancer cells in blood, reportedly capable of detecting one epithelial cell in 1 ml of peripheral blood.

A related aspect of the invention is directed to predicting susceptibility to developing cancer in an individual. In one embodiment, a method for predicting susceptibility to cancer comprises detecting TLR9 or TLR10 mRNA or protein in a tissue sample, its presence indicating susceptibility to cancer, wherein the degree of TLR9 or TLR10 mRNA expression present is proportional to the degree of susceptibility.

Yet another related aspect of the invention is directed to methods for assessment of tumor aggressiveness (Orlandi, et al., Cancer Res. 62:567 (2002)). In one embodiment, a method for gauging aggressiveness of a tumor comprises determining the level of TLR9 or TLR10 mRNA or protein expressed by cells in a sample of the tumor, comparing the level so determined to the level of TLR9 or TLR10 mRNA or protein expressed in a corresponding normal tissue taken from the same individual or a normal tissue reference sample, wherein the degree of TLR9 or TLR10 mRNA or protein expression in the tumor sample relative to the normal sample indicates the degree of aggressiveness.

Methods for detecting and quantifying the expression of TLR9 or TLR10 mRNA or protein are described herein and use standard nucleic acid and protein detection and quantification technologies well known in the art. Standard methods for the detection and quantification of TLR9 or TLR10 mRNA include in situ hybridization using labeled TLR9 or TLR10 riboprobes (Gemou-Engesaeth, et al., Pediatrics, 109:E24-E32 (2002)), Northern blot and related techniques using TLR9 or TLR10 polynucleotide probes (Kunzli, et al., Cancer 94:228 (2002)), RT-PCR analysis using primers specific for TLR9 or TLR10 (Angchaiskisiri, et al., Blood 99:130 (2002)), and other amplification type detection methods, such as, for example, branched DNA (Jardi, et al., J. Viral Hepat. 8:465-471 (2001)), SISBA, TMA (Kimura, et al., J. Clin. Microbiol. 40:439-445 (2002)), and microarray products of a variety of sorts, such as oligos, cDNAs, and monoclonal antibodies. In a specific embodiment, real-time RT-PCR may be used to detect and quantify TLR9 or TLR10 mRNA expression (Simpson, et al., Molec. Vision 6:178-183 (2000)). Standard methods for the detection and quantification of protein may be used for this purpose. In a specific embodiment, polyclonal-or monoclonal antibodies specifically reactive with the wild-type TLR9 or TLR10 may be used in an immunohistochemical assay of biopsied tissue (Ristimaki, et al., Cancer Res. 62:632 (2002)).

(2) Medical Imaging

TLR9 or TLR10 antibodies and fragments thereof are useful in medical imaging of sites expressing TLR9 or TLR10, respectively. Such methods involve chemical attachment of a labeling or imaging agent, such as a radioisotope, which include ⁶⁷Cu, ⁹⁰Y, ¹²⁵I, ¹³¹I, ¹⁸⁶Re, ¹⁸⁸Re, ²¹¹At, and ²¹²Bi, administration of the labeled antibody and fragment to a subject in a pharmaceutically acceptable carrier, and imaging the labeled antibody and fragment in vivo at the target site. Radiolabelled anti-TLR9 or anti-TLR10 antibodies or fragments thereof may be particularly useful in in vivo imaging of TLR9 or TLR10 expressing cancers, such as lymphomas or leukemias. Such antibodies may provide highly sensitive methods for detecting metastasis of TLR9- or TLR10-expressing cancers.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

### EXAMPLE 1

### Cell Lines of Lymphoma and Leukemia Origin Express High Levels of TLR9 mRNA

Expression of TLR9 was determined in various lymphoid and myeloid cell lines. Poly-A messenger RNA was isolated from the cell lines listed in Table 1 and subjected to quantitative, real-time PCR analysis (Simpson, et al., Molec. Vision. 6: 178-183 (2000)) to determine the relative copy number of TLR9 mRNA expressed per cell in each line. Elongation factor 1 mRNA expression was used as a positive control and normalization factors in all samples.

All assays were performed in duplicate with the resulting values averaged and expressed as “−” for samples with no detectable TLR 9 mRNA in that sample to “+++” for samples with the highest mRNA copy number for TLR9. The following quantitation scale for the real-time PCR experiments was used: “−”=0 copies/cell; “+”=approximately 1-10 copies/cell; “++”=approximately 11-50 copies/cell; and “+++”=approximately >50 copies/cell. The results are indicated in Table 1.

TABLE 1

| Cell Line | TLR9 mRNA Expression |
|---|---|
| Burkitt's Lymphoma (CA46) cell line | +++ |
| Diffuse Lymphoma (HT) cell line | +++ |
| B Lymphoma (RL) cell line | +++ |
| Burkitt's Lymphoma (GA-10) cell line | +++ |
| AML-193 Acute Myeloid Leukemia cell | ++ |
| line | |
| Acute Myeloid Leukemia (KG-1) cell line | + |
| Promyelomonocytic (HL-60) cell line | ++ |

The results shown in Table 1 show that the B cell lymphoma cell lines CA-46, RL, GA-10 and HT had high levels of expression. Additionally, the promyelomonocytic cell line HL-60 was observed to have medium expression levels, whereas the acute myeloid leukemia cell lines AML-193 and KG-1 were found to have moderate and low levels of mRNA expression respectively. These results demonstrate that TLR9 mRNA is highly expressed in cell lines derived from B cell lymphomas and myeloid leukemias.

### EXAMPLE 2

### TLR9 mRNA is Highly Expressed in Patient Tissues

Expression of TLR9 was determined in various healthy and tumor tissues (Table 2). Poly-A mRNA was isolated from the tonsilar lymph node and lymphoma, AML and Hodgkin's Disease tissue samples obtained from the Cooperative Human Tissue Network (CHTN, National Cancer Institute). All other RNAs were purchased from Clontech (Palo Alto, Calif.) and Ambion (Austin, Tex.). Tonsilar lymph nodes were used as non-lymphoma containing nodal tissue (7117), whereas 5348, 5856 and 6796 were B-cell follicular lymphomas and samples 22601 and 6879 were diffuse large B-cell lymphoma samples. Lymph node and lymphoma patient tissue samples were snap frozen immediately after surgical removal. Poly-A⁺ mRNA was subjected to quantitative, real-time PCR analysis, as described in Example 1, to determine the relative expression of TLR9 mRNA in the sample. All assays were performed in duplicate with the resulting values averaged and expressed as “−” for samples with no detectable TLR9 mRNA in that sample to “+++” for samples with the highest mRNA copy number for TLR9. The following quantitation scale for the real-time PCR experiments was used: “−”=0 copies/cell; “+”=approximately 1-10 copies/cell; “++”=approximately 11-50 copies/cell; and “+++”=approximately >50 copies/cell. The results are indicated in Table 2.

TABLE 2

| Patient Tissue | TLR9 mRNA Expression |
|---|---|
| Tonsilar lymph node 7117 | ++ |
| Follicullar Lymphoma 5348 | + |
| Follicullar Lymphoma 5856 | + |
| Diffuse Large B Cell Lymphoma 6879 | + |
| Diffuse Large B Cell Lymphoma 22601 | + |
| Follicular Lymphoma 6796 | ++ |
| T Cell Lymphoma 5664 | ++ |
| Hodgkin's Disease 566 | + |
| Acute Myeloid Leukemia 565 | + |
| Peripheral Blood B cells | ++ |
| Peripheral Blood Monocytes | + |
| Peripheral Blood T cells | − |
| Placenta tissue | + |
| brain tissue | + |
| Pancreas tissue | − |
| Lung tissue | ++ |
| Adrenal tissue | − |
| Prostate tissue | + |
| Thymus tissue | + |
| testis tissue | + |
| Small intestine tissue | + |
| colon tissue | − |

The results in Table 2 demonstrate that B cell and T cell lymphoma tissue expressed low to moderate levels of TLR9 mRNA. Additionally, low levels of expression were also observed in Hodgkin's disease and AML tissue. Non-cancerous tonsilar lymph nodes, healthy peripheral blood B cells (CD19+ cells), and lung tissue were found to have medium levels of expression. Healthy peripheral blood monocytes (CD-14+) showed low levels of expression. Most non-hematopoeitic healthy tissues did not demonstrate expression of TLR9 or only expressed at low levels. The results demonstrate TLR9 mRNA expression in different Non-Hodgkin's B cell lymphoma, T cell lymphomas, Hodgkin's disease and AML tissues and cell lines, and indicate that TLR9 may be used as an immunotherapeutic antibody target or as a diagnostic marker for these types of disorders.

### EXAMPLE 3

### Diagnostic Methods Using TLR9-Specific Antibodies to Detect TLR9 Expression

Expression of TLR9 in tissue samples (normal or diseased) was detected using anti-TLR9 antibodies (see Table 3). Samples were prepared for immunohistochemical (IHC) analysis by fixing tissues in 10% formalin embedding in paraffin, and sectioning using standard techniques. Sections were stained using the TLR9-specific antibody followed by incubation with a secondary horse radish peroxidase (HRP)-conjugated antibody and visualized by the product of the HRP enzymatic reaction. Data as seen in Table 3 shows that TLR9 is highly expressed on cell surface of tumor tissues. No expression of TLR9 was observed on the cell surface of normal tissues. In addition to cell surface expression of TLR9 on leukemia and lymphoma tissues, TLR9 expression was found in solid tumors of prostate, breast, colon, and squamous cell carcinoma (see Table 4). Based on this expression pattern, it is likely that other cancers of epithelial and squamous cell origin will also express TLR9.

TABLE 3

| Tissue | Positive | Total |
|---|---|---|
| Acute myelogenous leukemia | 18 | 23 |
| Acute myelomonocytic leukemia | 9 | 11 |
| Acute lymphoblastic leukemia | 4 | 4 |
| Chronic myelogenous leukemia | 10 | 13 |
| Chronic lymphocytic leukemia | 13 | 15 |
| B cell large cell lymphoma | 6 | 7 |
| Malignant lymphoma | 7 | 8 |
| Acute leukemia | 1 | 2 |
| Lymphosarcoma cell leukemia | 2 | 2 |
| Normal prostate | 0 | 4 |
| Normal breast | 0 | 4 |
| Normal colon | 0 | 4 |
| Normal thyroid | 0 | 4 |
| Normal adrenal gland | 0 | 4 |
| Normal placenta | 0 | 4 |
| Normal tonsil | 0 | 4 |
| Normal lymph node | 0 | 4 |
| Normal spleen | 0 | 4 |
| Normal heart | 0 | 4 |
| Normal skeletal muscle | 0 | 4 |
| Normal liver | 0 | 4 |
| Normal pancreas | 0 | 4 |
| Normal ovary | 0 | 4 |
| Normal myometrium | 0 | 4 |
| Normal endometrium | 0 | 4 |
| Normal endocervical | 0 | 4 |
| Normal kidney | 0 | 4 |
| Normal seminal vesicle | 0 | 4 |
| Normal testis | 0 | 4 |
| Normal brain | 0 | 4 |
| Normal lung | 0 | 4 |
| Normal cerebellum | 0 | 4 |
| Normal cerebrum | 0 | 4 |

TABLE 4

| Tumor Tissue | TLR9 Expression |
|---|---|
| Prostate, Gleason scores 7, 8 and 9 | +++ |
| Breast, stage 3 and 4 | +++ |
| Colon, Dukes B and C | +++ |
| Squamous cell carcinoma | +++ |

IHC analysis was also performed on tissues derived from autoimmune disorders. Table 5 shows that TLR9 was overexpressed in tissues from systemic lupus erythematosus, Hasimoto thyroiditis, Sjörgen's syndrome, and pericarditis lupus.

TABLE 5

| Tissue | Positive | Total |
|---|---|---|
| Systemic lupus erythematosus | 17 | 20 |
| Hasimoto thyroiditis | 12 | 15 |
| Sjörgen's syndrome | 2 | 5 |
| Pericarditis lupus | 13 | 17 |

IHC analysis was also performed on tissues derived from rejected organ transplants. Table 6 shows that TLR9 was overexpressed in rejected heart, liver, and kidney, whereas TLR9 was not present on healthy tissues.

TABLE 6

| Tissue | Positive | Total |
|---|---|---|
| Rejected heart | 23 | 27 |
| Rejected liver | 31 | 35 |
| Rejected kidney | 15 | 18 |
| Normal heart | 0 | 4 |
| Normal liver | 0 | 4 |
| Normal kidney | 0 | 4 |

Expression of TLR9 on the surface of cells within a blood sample is detected by flow cytometry. Peripheral blood mononuclear cells (PBMC) are isolated from a blood sample using standard techniques. The cells are washed with ice-cold PBS and incubated on ice with the TLR9-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument settings are determined using FACS-Brite calibration beads (Becton-Dickinson).

Tumors expressing TLR9 are imaged using TLR9-specific antibodies conjugated to a radionuclide, such as ¹²³I, and injected into the patient for targeting to the tumor followed by X-ray or magnetic resonance imaging.

### EXAMPLE 4

### TLR10 is Expressed in Cell Lines of Lymphoma Origin

Expression of TLR10 was determined in various lymphoid and myeloid cell lines. Poly-A messenger RNA was isolated from the cell lines listed in Table 7 and subjected to quantitative, real-time PCR analysis (Simpson et al., Molec. Vision 6:178-183 (2000)) to determine the relative copy number of TLR10 mRNA expressed per cell in each line. Elongation factor 1 mRNA expression was used as a positive control and normalization factor in all samples.

All assays were performed in duplicate with the resulting values averaged and expressed as “−” for samples with no detectable TLR10 mRNA in that sample to “+++” for samples with the highest mRNA copy number for TLR10. The following estimated quantitation scale for the real-time PCR experiments was used: “−”=0 copies/cell; “+”=approximately 1-10 copies/cell; “++”=approximately 11-50 copies/cell; and “+++”=approximately >50 copies/cell. The results are indicated in Table 7.

TABLE 7

| Cell Line | TLR10 mRNA Expression |
|---|---|
| Burkitt's Lymphoma (CA-46) | ++ |
| B cell Lymphoma (RA1) | +++ |
| Diffuse B cell Lymphoma (HT) | ++ |
| B cell Lymphoma (DB) | ++ |
| B cell Lymphoma (RL) | ++ |
| Acute Myeloid Leukemia (AML-193) | − |
| Pro-myelomonocitic Leukemia (HL-60) | − |
| Multiple Myeloma (RPMI) | − |

The results shown in Table 7 demonstrate that the B cell lymphoma cell lines CA-46 and RA1 had high levels of TLR10 expression. Additionally, the B cell lymphoma cell lines HT, DB, and RL were observed to have medium TLR10 expression levels, whereas the myeloid cell lines had no observed TLR10 expression. These results demonstrate that TLR10 mRNA is highly expressed in cell lines derived from B cell lymphomas.

### EXAMPLE 5

### TLR10 mRNA is Expressed in Patient Tissues of Lymphoma Origin

Expression of TLR10 was determined in various healthy and tumor tissues (Table 8). Poly-A mRNA was isolated from the lymphoma and acute myeloid leukemia tissue samples obtained from the Cooperative Human Tissue Network (CHTN, National Cancer Institute). All other RNAs were purchased from Clontech (Palo Alto, Calif.), Ambion (Austin, Tex.), and Stratagene (La Jolla, Calif.). Tissues 5348 and 5856 were B cell follicular lymphomas and samples 22601 and 6879 were diffuse large B cell lymphoma samples. Lymphoma patient tissue samples were snap frozen immediately after surgical removal. Poly-A⁺ mRNA was subjected to quantitative, real-time PCR analysis, as described in Examples 1 and 3, to determine the relative expression of TLR10 mRNA in the sample. All assays were performed in duplicate with the resulting values averaged and expressed as “−” for samples with no detectable TLR10 mRNA in that sample to “+++” for samples with the highest mRNA copy number for TLR10. The following quantitation scale for the real-time PCR experiments was used: “−”=0 copies/cell; “+”=approximately 1-10 copeis/cell; “++”=approximately 11-50 copies/cell; and “+++”=approximately >50 copies/cell. The results are indicated in Table 8.

TABLE 8

| Patient Tissue | TLR10 mRNA Expression |
|---|---|
| Follicular Lymphoma 5348 | ++ |
| Follicular Lymphoma 5856 | ++ |
| Diffuse Large B cell Lymphoma 6879 | ++ |
| Diffuse Large B cell Lymphoma 22601 | ++ |
| Anaplastic Large T cell Lymphoma 5664 | ++ |
| Acute Myeloid Leukemia 565 | + |
| Normal Ileum | + |
| Ileum B cell lymphoma | +++ |
| CD14+ cells | + |
| Adult spleen tissue | ++ |
| Thymus tissue | + |
| Testicle tissue | − |
| Small Intestine tissue | ++ |
| Adult brain tissue | + |
| Adult liver tissue | − |
| Heart tissue | − |
| Kidney tissue | + |
| Lung tissue | + |
| Prostate tissue | − |
| Adrenal tissue | − |
| Bladder tissue | − |
| Breast tissue | + |
| Cervix tissue | + |
| Colon tissue | − |
| Skeletal muscle tissue | + |
| Mammary gland tissue | − |
| Ovary tissue | − |
| Pancreas tissue | − |
| Placenta tissue | − |

The results in Table 8 demonstrate that B cell and T cell lymphoma tissue expressed moderate to high levels of TLR10 mRNA. Additionally, ileum B cell lymphoma tissue expressed high levels of TLR10 mRNA as compared to normal ileum tissue. All other non-cancerous and healthy tissues showed low levels of or no TLR10 expression. The results demonstrate TLR10 mRNA expression in different Non-Hodgkin's B cell lymphomas, T cell lymphoma, acute myeloid leukemia, and ileum B cell lymphoma tissues and indicate that TLR10 may be used as an immunotherapeutic antibody target or as a diagnostic marker for these types of disorders.

### EXAMPLE 6

### Diagnostic Methods using TLR10-Specific Antibodies to Detect TLR10 Expression

Expression of TLR10 in tissue samples (normal or diseased) was detected using 15 anti-TLR10 antibodies (see Table 9). Samples were prepared for immunohistochemical (IHC) analysis (Clinomics Biosciences, Inc., Pittsfield, Mass.) by fixing tissues in 10% formalin embedding in paraffin, and sectioning using standard techniques. Sections were stained using the TLR10-specific antibody followed by incubation with a secondary horse radish peroxidase (HRP)-conjugated antibody and visualized by the product of the HRP enzymatic reaction. Data as seen in Table 9 shows that TLR10 is highly expressed on cell surface of tumor tissues. No expression of TLR 10 was observed on the cell surface of normal tissues; however a low level of TLR10 expression could be detected in tonsils. In addition to TLR10 expression in leukemia and lymphoma tissues, TLR10 expression was found in solid tumors from prostate, breast, colon, and squamous cell carcinoma (see Table 10). Based on this expression pattern, it is likely that other cancers of epithelial and squamous cell origin will also express TLR10.

TABLE 9

| Tissue | Positive | Total |
|---|---|---|
| Acute myelogenous leukemia | 19 | 23 |
| Acute myelomonocytic leukemia | 11 | 11 |
| Acute lymphoblastic leukemia | 3 | 4 |
| Chronic myelogenous leukemia | 6 | 13 |
| Chronic lymphocytic leukemia | 13 | 15 |
| B cell large cell lymphoma | 3 | 7 |
| Malignant lymphoma | 6 | 8 |
| Acute leukemia | 1 | 2 |
| Lymphosarcoma cell leukemia | 1 | 2 |
| Normal prostate | 0 | 4 |
| Normal breast | 0 | 4 |
| Normal colon | 0 | 4 |
| Normal thyroid | 0 | 4 |
| Normal adrenal gland | 0 | 4 |
| Normal placenta | 0 | 4 |
| Normal tonsil | 0 | 4 |
| Normal lymph node | 0 | 4 |
| Normal spleen | 0 | 4 |
| Normal heart | 0 | 4 |
| Normal skeletal muscle | 0 | 4 |
| Normal liver | 0 | 4 |
| Normal pancreas | 0 | 4 |
| Normal ovary | 0 | 4 |
| Normal myometrium | 0 | 4 |
| Normal endometrium | 0 | 4 |
| Normal endocervical | 0 | 4 |
| Normal kidney | 0 | 4 |
| Normal seminal vesicle | 0 | 4 |
| Normal testis | 0 | 4 |
| Normal brain | 0 | 4 |
| Normal lung | 0 | 4 |
| Normal cerebellum | 0 | 4 |
| Normal cerebrum | 0 | 4 |

TABLE 10

| Tumor Tissue | TLR9 Expression |
|---|---|
| Prostate, Gleason scores 7, 8 and 9 | +++ |
| Breast, stage 3 and 4 | +++ |
| Colon, Dukes B and C | +++ |
| Squamous cell carcinoma | +++ |

IHC analysis was also performed on tissues derived from autoimmune diseases. Table 11 shows that TLR10 was overexpressed in systemic lupus erythematosus, Hasimoto thyroiditis, Sjörgen's syndrome, and pericarditis lupus.

TABLE 11

| Tissue | Positive | Total |
|---|---|---|
| Systemic lupus erythematosus | 18 | 20 |
| Hasimoto thyroiditis | 14 | 15 |
| Sjörgen's syndrome | 5 | 5 |
| Pericarditis lupus | 17 | 17 |

IHC was also performed on tissues derived from rejected organ transplants. Table 12 shows that TLR10 was overexpressed in rejected heart, liver, and kidney, whereas TLR10 was not present on healthy tissues.

TABLE 12

| Tissue | Positive | Total |
|---|---|---|
| Rejected heart | 24 | 27 |
| Rejected liver | 30 | 35 |
| Rejected kidney | 13 | 18 |
| Normal heart | 0 | 4 |
| Normal liver | 0 | 4 |
| Normal kidney | 0 | 4 |

Expression of TLR10 on the surface of cells within a blood sample is detected by flow cytometry. Peripheral blood mononuclear cells (PBMC) are isolated from a blood sample using standard techniques. The cells are washed with ice-cold PBS and incubated on ice with the TLR10-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument settings are determined using FACS-Brite calibration beads (Becton-Dickinson).

Tumors expressing TLR10 are imaged using TLR10-specific antibodies conjugated to a radionuclide, such as ¹²³I, and injected into the patient for targeting to the tumor followed by X-ray or magnetic resonance imaging.

### EXAMPLE 7

### Production of TLR9- or TLR10-Specific Antibodies

Cells expressing TLR9 or TLR10 are identified using antibodies to TLR9 or TLR10, respectively. Polyclonal antibodies are produced by DNA vaccination or by injection of peptide antigens into rabbits or other hosts. An animal, such as a rabbit, is immunized with a peptide from the extracellular region of TLR9 or TLR10 conjugated to a carrier protein, such as BSA (bovine serum albumin) or KLH (keyhole limpet hemocyanin). The rabbit is initially immunized with conjugated peptide in complete Freund's adjuvant, followed by a booster shot every two weeks with injections of conjugated peptide in incomplete Freund's adjuvant. Anti-TLR9 or anti-TLR10 antibodies are affinity purified from rabbit serum using a TLR9 or TLR10 peptide, respectively, coupled to Affi-Gel 10 (Bio-Rad), and stored in phosphate-buffered saline (PBS) with 0.1% sodium azide. To determine that the polyclonal antibodies are TLR9- or TLR10-specific, an expression vector encoding TLR9 or TLR10, respectively, is introduced into mammalian cells. Western blot analysis of protein extracts of non-transfected cells and the TLR9- or TLR10-containing cells is performed using the polyclonal antibody sample as the primary antibody and a horseradish peroxidase-labeled anti-rabbit antibody as the secondary antibody. Detection of an approximately 115 kD or 89 kD band in the TLR9- or TLR10-containing cells, respectively, and lack thereof in the control cells indicates that the polyclonal antibodies are specific for TLR9 or TLR10.

Monoclonal antibodies are produced by injecting mice with a TLR9 or TLR10 peptide, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### EXAMPLE 8

### In Vitro Antibody-Dependent Cytotoxicity Assay

The ability of a TLR9- or TLR10-specific antibody to induce antibody-dependent cell-mediated cytoxicity (ADCC) is determined in vitro. ADCC is performed using the CytoTox 96 Non-Radioactive Cytoxicity Assay (Promega; Madison, Wis.) (Hornick et al., Blood 89:4437-4447, (1997)) as well as effector and target cells. Peripheral blood mononuclear cells (PBMC) or neutrophilic polymorphonuclear leukocytes (PMN) are two examples of effector cells that can be used in this assay. PBMC are isolated from healthy human donors by Ficoll-Paque gradient centrifugation, and PMN are purified by centrifugation through a discontinuous percoll gradient (70% and 62%) followed by hypotonic lysis to remove residual erythrocytes. RA1 B cell lymphoma cells (for example) are used as target cells.

RA1 cells are suspended in RPMI 1640 medium supplemented with 2% fetal bovine serum and plated in 96-well V-bottom microtitier plates at 2×10⁴ cells/well. TLR9- or TLR10-specific antibodies are added in triplicate to individual wells at 1 μg/ml, and effector cells are added at various effector:target cell ratios (12.5:1 to 50:1). The plates are incubated for 4 hours at 37° C. The supernatants are then harvested, lactate dehydrogenase release determined, and percent specific lysis calculated using the manufacture's protocols.

### EXAMPLE 9

### Toxin-Conjugated TLR9- or TLR10-Specific Antibodies

Antibodies to TLR9 or TLR10 are conjugated to toxins and the effect of such conjugates in animal models of cancer is evaluated. Chemotherapeutic agents, such as calicheamycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets TLR9- or TLR10-expressing cells, such as B cell lymphomas, and deliver the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated TLR9- or TLR10-specific antibodies is evaluated in vitro, for example, by incubating TLR9- or TLR10-expressing target cells (such as the RA1 B cell lymphoma cell line) with various concentrations of conjugated antibody, medium alone, carboplatin alone, or antibody alone. The antibody-toxin conjugate specifically targets and kills cells bearing the TLR9 or TLR10 antigen, whereas, cells not bearing the antigen, or cells treated with medium alone, carboplatin alone, or antibody alone, show no cytotoxicity.

The antitumor efficacy of carboplatin-conjugated TLR9- or TLR10-specific antibodies is demonstrated in in vivo murine tumor models. Five to six week old, athymic nude mice are engrafted with tumors subcutaneously or through intravenous injection. Mice are treated with the TLR9- or TLR10-carboplatin conjugate or with a non-specific antibody-carboplatin conjugate. Tumor xenografts in the mouse bearing the TLR9 or TLR10 antigen are targeted and bound to by the TLR9- or TLR10-carboplatin conjugate, respectively. This results in tumor cell killing as evidenced by tumor necrosis, tumor shrinkage, and increased survival of the treated mice.

Other toxins are conjugated to TLR9- or TLR 10-specific antibodies using methods known in the art. An example of a toxin conjugated antibody in human clinical trials is CMA-676, an antibody to -the CD33 antigen in AML which is conjugated with calicheamicin toxin (Larson, Semin. Hematol. 38(Suppl 6):24-31 (2001)).

### EXAMPLE 10

### Radio-Immunotherapy Using TLR9- or TLR10-Specific Antibodies

Animal models are used to assess the effect of antibodies specific to TLR9 or TLR10 as vectors in the delivery of radionuclides in radio-immunotherapy to treat lymphoma, hematological malignancies, and solid tumors. Human tumors are propagated in 5-6 week old athymic nude mice by injecting a carcinoma cell line or tumor cells subcutaneously. Tumor-bearing animals are injected intravenously with radio-labeled anti-TLR9 or anti-TLR10 antibody (labeled with 30-40 μCi of ¹³¹I, for example) (Behr, et al., Int. J. Cancer 77: 787-795 (1988)). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### EXAMPLE 11

### Immunotherapy Using TLR9- or TLR10-Specific Antigodies

Animal models are used to evaluate the effect of TLR9- or TLR10-specific antibodies as targets for antibody-based immunotherapy using monoclonal antibodies. Human myeloma cells are injected into the tail vein of 5-6 week old nude mice whose natural killer cells have been eradicated. To evaluate the ability of TLR9- or TLR10-specific antibodies in preventing tumor growth, mice receive an intraperitoneal injection with TLR9- or TLR10-specific antibodies either 1 or 15 days after tumor inoculation followed by either a daily dose of 20 μg or 100 μg once or twice a week, respectively (Ozaki, et al., Blood 90:3179-3186 (1997)). Levels of human IgG (from the immune reaction caused by the human tumor cells) are measured in the murine sera by ELISA.

The effect of TLR9- or TLR10-specific antibodies on the proliferation of myeloma cells is examined in vitro using a ³H-thymidine incorporation assay (Ozaki et al., supra). Cells are cultured in 96-well plates at 1×10⁵ cells/ml in 100 μl/well and incubated with various amounts of TLR9 or TLR10 antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the TLR9 or TLR10 monoclonal antibody is examined by the effect of complements on myeloma cells using a ⁵¹Cr-release assay (Ozaki et al., supra). Myeloma cells are labeled with 0.1 mCi ⁵¹Cr-sodium chromate at 37° C. for 1 h. ⁵¹Cr-labeled cells are incubated with various concentrations of TLR9 or TLR10 monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1% NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the TLR9 or TLR10 monoclonal antibody is measured using a standard 4 h ⁵¹Cr-release assay (Ozaki et al., supra). Splenic mononuclear cells from SCID mice are used as effector cells and cultured with or without recombinant interleukin-2 (for example) for 6 days. ⁵¹Cr-labeled target myeloma cells (1×10⁴ cells) are placed in 96-well plates with various concentrations of anti-TLR9 or anti-TLR10 monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### EXAMPLE 12

### TLR9- or TLR10-Specific Antibodies as Immunosuppressants

Animal models are used to assess the effect of TLR9- or TLR10-specific antibodies on blocking signaling through the TLR9 or TLR10 receptor to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies (Yang, et al., Int. Immunopharm. 2:389-397 (2002)). Animals are divided into five groups, three of which are injected with anti-TLR9 or anti-TLR10 antibodies for 10 days, and 2 of which receive no treatment. Two of the experimental groups and one control group are injected with either Earle's balanced salt solution (EBSS) containing 5-10×10⁷ HRBCs or EBSS alone. Anti-TLR9 or anti-TLR10 antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosupression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm using p-nitrophenyl phosphate as a substrate.

Lymphocyte proliferation is measured in response to the T and B cell activators concanavalin A and lipopolysaccharide, respectively (Jiang, et al., J. Immunol. 154:3138-3146 (1995). Mice are randomly divided into 2 groups, 1 receiving anti-TLR9 or anti-TLR10 antibody therapy for 7 days and 1 as a control. At the end of the treatment, the animals are sacrificed by cervical dislocation, the spleens are removed, and splenocyte suspensions are prepared as above. For the ex vivo test, the same number of splenocytes are used, whereas for the in vivo test, the anti-TLR9 or anti-TLR10 antibody is added to the medium at the beginning of the experiment. Cell proliferation is also assayed using the ³H-thymidine incorporation assay described above (Ozaki, et al., Blood 90: 3179 (1997)).

### EXAMPLE 13

### Cytokine Secretion in Response to TLR9 or TLR10 Peptide Fragments

Assays are carried out to assess activity of fragments of the TLR9 or TLR10 protein, such as the Ig domain, to stimulate cytokine secretion and to stimulate immune responses in NK cells, B cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of the TLR9 or TLR10 that block activation through the TLR9 or TLR10 receptor, respectively, may be used to block immune stimulation in natural killer (NK), B, T, and myeloid cells.

Fusion proteins containing fragments of the TLR9 or TLR10, such as the Ig domain (TLR9-Ig or TLR10-Ig, respectively), are made by inserting a CD33 leader peptide, followed by a TLR9 or TLR10 domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ (IFN-γ) secretion assays (Martin, et al., J. Immunol. 167:3668-3676 (2001)).

PBMCs are activated with a suboptimal concentration of soluble CD3 and various concentrations of purified, soluble anti-TLR9 or anti-TLR10 monoclonal antibody or control IgG. For TLR9-Ig or TLR10-Ig cytokine assays, anti-human Fc Ig at 5 or 20 μg/ml is bound to 96-well plates and incubated overnight at 4° C. Excess antibody is removed and either TLR9-Ig or TLR10-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and IFN-γ levels are measured by sandwich ELISA, using primary and biotinylated secondary anti-human IFN-γ antibodies as recommended by the manufacturer.

### EXAMPLE 14

### Tumor Imaging Using TLR9- or TLR10-Specific Antibodies

TLR9- or TLR10-specific antibodies are used for imaging TLR9- or TLR10-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh. When the tumors reach approximately 1 cm in diameter, the mice are injected intravenously with an inoculum containing 100 μCi/10 μg of ¹³¹I-labeled TLR9- or TLR10-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.) (Hornick, et al., Blood 89:4437-4447 (1997)).

We claim:

1. A pharmaceutical composition comprising an anti-Toll-like Receptor, selected from the group consisting of Toll-like receptor 9 (TLR9) and Toll-like receptor 10 (TLR10), antibody specific for cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, wherein said antibody specifically binds to a polypeptide having an amino acid sequence of SEQ ID NO: 4 or amino acid 20 to amino acid 811 of SEQ ID NO: 4.

2. The pharmaceutical composition of claim 1, wherein said antibody is a monoclonal anti-TLR9 antibody or fragment thereof.

3. The pharmaceutical composition of claim 1, wherein said antibody is a monoclonal anti-TLR10 antibody or fragment thereof.

4. The pharmaceutical composition of claim 1, wherein said antibody is administered in an amount effective to kill or inhibit the growth of cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs.

5. A method of targeting a TLR protein selected from the group consisting of TLR9 and TLR10, on cells that cause disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, comprising the step of administering a composition to said cells in an amount effective to target said TLR-expressing cells, wherein said composition is an anti-TLR antibody that specifically binds to a polypeptide selected from the group consisting of having an amino acid sequence of SEQ ID NO: 2, amino acid 25 to amino acid 811 of SEQ ID NO: 2, SEQ ID NO: 4, and amino acid 20 to amino acid 811 of SEQ ID NO: 4.

6. A method of killing or inhibiting the growth of TLR-expressing cells, selected from the group of TLR9 and TLR10, that cause disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, comprising the step of administering a composition to said cells in an amount effective to kill or inhibit the growth of said cells, wherein said composition is an anti-TLR antibody that specifically binds to a polypeptide selected from the group consisting of having an amino acid sequence of SEQ ID NO: 2, amino acid 25 to amino acid 818 of SEQ ID NO: 2, SEQ ID. NO: 4, and amino acid 20 to amino acid 811 of SEQ ID NO: 4.

7. A method of killing or inhibiting the growth of TLR-expressing cells, selected from the group of TLR9 and TLR10, that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, comprising the step of administering a vaccine to said cells in an amount effective to kill or inhibit the growth of said cancer cells, wherein said vaccine comprises a TLR polypeptide selected from the group consisting of having an amino acid sequence of SEQ ID NO: 2, amino acid 25 to amino acid 811 of SEQ ID NO: 2, SEQ ID NO: 4, and amino acid 20 to amino acid 811 of SEQ ID NO: 4, or immunogenic fragment thereof.

8. A method of killing or inhibiting the growth of TLR-expressing cells, selected from the group consisting of TLR9 and TLR10, that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, comprising the step of administering a composition to said cells in an amount effective to kill or inhibit the growth of said cells, wherein said composition comprises a nucleic acid selected from the group consisting of SEQ ID NO: 1 encoding TLR9, and SEQ ID NO: 3 encoding TLR10, or immunogenic fragment thereof, within a recombinant vector.

9. A method of killing or inhibiting the growth of TLR-expressing cells, selected from the group consisting of TLR9 and TLR10, that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, comprising the step of administering a composition to said cells in an amount effective to kill or inhibit the growth of said cells, wherein said composition comprises an antigen-presenting cell comprising a nucleic acid selected from the group consisting of SEQ ID NO: 1 encoding TLR9, and SEQ ID NO: 3 encoding TLR10, or immunogenic fragment thereof, within a recombinant vector.

10. The method according to any one of claims 5-9, wherein said cells are contacted with as second therapeutic agent.

11. The method according to any of claims 5-6, wherein said anti-TLR antibody composition is administered in an amount effective to achieve a dosage range from about 0.1 to about 10 mg/kg body weight.

12. The method according to any one of claims 5-9, wherein said pharmaceutical composition is administered in a sterile preparation together with a pharmaceutically acceptable carrier therefore.

13. A method of diagnosing a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organscomprising the steps of:
(a) detecting or measuring the expression of TLR protein, selected from the group consisting of TLR9 and TLR10 on a cell; and
(b) comparing said expression to a standard indicative of said disease.

14. The method according to claim 13, wherein said expression is TLR9 mRNA expression.

15. The method according to claim 13, wherein said expression is TLR10 mRNA expression.

16. The method according to claim 13, wherein said expression is detected or measured using anti-TLR9 antibodies.

17. The method according to claim 13, wherein said expression is detected or measured using anti-TLR10 antibodies.

18. Use of an anti-TLR antibody, selected from the group consisting of anti-TLR9 and anti-TLR10, in preparation of a medicament for killing or inhibiting the growth of TLR9- or TLR10-expressing cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs, wherein said antibody specifically binds to a polypeptide having the amino acid sequence selected from the group consisting of SEQ ID NO: 2, amino acid 25 to amino acid 818 of SEQ ID NO: 2, SEQ ID NO: 4, and amino acid 20 to amino acid 811 of SEQ ID NO: 4.

19. Use of a polypeptide having an amino acid sequence selected from the group consisting of SEQ ID NO: 2, amino acid 25 to amino acid 818 of SEQ ID NO: 2, SEQ ID NO: 4, and amino acid 20 to amino acid 811 of SEQ ID NO: 4 in preparation of a vaccine for killing or inhibiting the growth of TLR9- or TLR10-expressing cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs.

20. Use of a nucleic acid selected from the group consisting of SEQ ID NO: 1 encoding TLR9, and SEQ ID NO: 3 encoding TLR10, or immunogenic fragment thereof, within a recombinant vector, in preparation of a medicament for killing or inhibiting the growth of TLR9- or TLR10-expressing cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs.

21. Use of an antigen-presenting cell comprising a nucleic acid selected from the group consisting of SEQ ID NO: 1 encoding TLR9, and SEQ ID NO: 3 encoding TLR10 or immunogenic fragment thereof, within a recombinant vector, in preparation of a medicament for killing or inhibiting the growth of TLR9- or TLR10-expressing cells that cause a disease selected from the group consisting of B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, solid phase cancer, autoimmune disorder, and rejection of transplanted tissues or organs.

22. An antibody that specifically binds to a polypeptide selected from the group consisting of having the amino acid sequence of SEQ ID NO: 2, amino acid 25 to amino acid 818 of SEQ ID NO: 2, SEQ ID NO: 4, and amino acid 20 to amino acid 811 of SEQ ID NO: 4.

23. The antibody of claim 22, wherein said antibody is a monoclonal anti-TLR9 antibody or fragment thereof.

24. The antibody of claim 22, wherein said antibody is a monoclonal anti-TLR10 antibody or fragment thereof.

25. The antibody of claim 22, wherein said antibody is labeled with a radioisotope.


SEQUENCE LISTING (4 sequences)

SEQ ID NO: 1 - DNA, 3099 bp, Homo sapiens
  CDS (1)..(3099)
atgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatag

SEQ ID NO: 2 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 3 - DNA, 2436 bp, Homo sapiens
  CDS (1)..(2436)
atgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaattaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattttactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataa

SEQ ID NO: 4 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGIRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL